(19) United States
(12) Patent Application Publication
(10) Pub. No.: US 2009/0047226 A1
(43) Pub. Date: Feb. 19, 2009

(54) Quick-drying cosmetic emulsions for roll-on application

(76) Inventors: Gertraud TECKENBROCK, Sprockhovel (DE); Barbara HEIDE, Krefeld (DE)

Correspondence Address: PAUL & PAUL, 2000 MARKET STREET, Suite 2900, PHILADELPHIA, PA, 19103-3229, US

(21) Appl. No.: 12/243,546

(22) Filed: Oct. 1, 2008

Related U.S. Application Data
(63) Continuation of application No. PCT/EP2007/003574, filed on Apr. 24, 2007.

(30) Foreign Application Priority Data
Apr. 28, 2006 (DE) 10 2006 020 382.8

(51) Int. Cl. A61K 8/37; A61Q 15/00

(52) U.S. Cl. 424/59; 424/65

(57) ABSTRACT

Cosmetic oil-in-water emulsions that are suitable in particular for application with a roll-on applicator and that exhibit high shelf stability, a non-greasy skin feel, and particularly quick drying characteristics, the emulsions containing a low concentration of oil phase or fat phase as well as at least one polysaccharide.

### CROSS-REFERENCE TO RELATED APPLICATIONS

This application is a continuation under 35 U.S.C. Sections 365(c) and 35 U.S.C. Section 120 of International Application No. PCT/EP2007/003574, filed Apr. 24, 2007. This application also claims priority under 35 U.S.C. Section 119 of German Patent Application No. DE 10 2006 020 382.8, filed Apr. 28, 2006. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present application relates to cosmetic oil-in-water emulsions that are suitable in particular for application with a roll-on applicator and exhibit high shelf stability, a non-greasy skin feel, and particularly quick drying characteristics, the emulsions containing a low concentration of oil phase or fat phase as well as at least one polysaccharide.

There are numerous possibilities for applying cosmetic compositions for skin and body care onto the skin. Cremes, salves, and lotions are usually removed from a jar, a tube, or a pump dispenser, and applied by hand and rubbed in. Dimensionally stable stick compounds are wiped over the skin from a stick dispenser until an effective quantity has been applied. Gels and cremes can also be applied with stick-like dispensers that are wiped over the skin with a dispenser surface. Numerous different application forms have been developed in particular for perspiration-inhibiting and/or deodorizing compositions for the underarm region, especially (in addition to those already recited) the sprays that contain and are free of propellant gases, and the roll-on compositions. In the latter, a slightly thickened liquid is applied from a reservoir container via a rotatably mounted ball by being rolled over the skin. Roll-on applicators are used chiefly for the underarm region, but are also suitable in principle for the care of the facial skin and of the body. For facial care, roll-on applicators of small dimensions serve in particular for the application of more highly concentrated active-substance sera onto selected problem areas, e.g., anti-wrinkle products for the corners of the eyes, the forehead, or the upper-lip region; anti-acne products; and anti-pimple products. This ensures efficient use (including in economic terms) of the valuable active substances. In addition, the use of more highly concentrated active substances that might generate an unpleasant skin feel over a large area (e.g., the anti-pimple active substance salicylic acid) can thereby be locally restricted. At the same time, the applicator permits convenient, time-saving application. Many cosmetic active substances are water-soluble, and their release on the skin can be delayed by oil and fat constituents of the cosmetic. As a purely aqueous solution, however, the product would be very difficult to meter and would thus be unacceptable to the consumer. Slight thickening, however, allows such a composition to be used conveniently with a roll-on applicator. Polymeric thickening agents are often used. A disadvantage in this context is that most polymeric thickening agents, in the requisite concentrations, generate a very sticky skin feel. In addition, many of these thickeners do not exhibit any additional cosmetic care effects. One advantageous alternative thereto is represented by emulsions having a low oil and fat content. Even without polymer thickeners, emulsification results in a rise in viscosity. The oil and fat portion of the emulsion furthermore exerts a skin-care effect.

Emulsions, in contrast to microemulsions, are thermodynamically unstable. The thermodynamically stable microemulsions can usually be stabilized only by means of a relatively high emulsifier content. A high concentration of emulsifiers can, however, in the least favorable case, have a skin-irritating effect and is, therefore, avoided whenever possible. In addition, microemulsions often form only in a very narrow mixing range between the individual components. For cosmetic compositions having multiple constituents, it can, therefore, on occasion be very difficult in terms of development engineering to establish suitable microemulsion ranges. Emulsions are stable for a certain time, since coalescence of the dispersed droplets is kinetically inhibited. This kinetic inhibition can be nullified by storage at high temperatures (relevant especially for production and marketing in hot countries) or in a context of storage with large temperature fluctuations (e.g., in insufficiently climate-controlled sales premises, during transport over longer distances). The high salt concentration in antiperspirant compositions, resulting from the relatively highly concentration of perspiration-inhibiting active substances, can also promote emulsion destabilization (e.g., due to salting-out effects).

In the case of roll-on emulsions having a typically large proportion of water, the moist skin feel directly after application can be perceived by the consumer as unpleasant.

(2) Description of Related Art, Including Information Disclosed Under 37 C.F.R. Sections 1.97 and 1.98.

EP 270328 A2 discloses perspiration-inhibiting oil-in-water emulsions that using a high concentration of polysaccharides, achieve encapsulation of the perfume oils that are contained. The high polysaccharide content can, however, have an unfavorable effect especially on skin feel. In addition, the high polysaccharide content can also impair the shelf stability of the emulsions, in particular at higher storage temperatures of 45° C. and above.

U.S. Pat. No. 4,499,069 discloses perspiration-inhibiting oil-in-water emulsions that contain approximately 22 wt % of an oil phase encompassing volatile silicone oils and PPG-15 stearyl ether, Steareth-2, Steareth-21, and 2 wt % aluminum starch octenylsuccinate. These emulsions are referred to as “shelf-stable,” but it is also indicated that they exhibit creaming of the dispersed phase after four weeks of storage at 45° C. This stability behavior is no longer sufficient for present-day consumer demands.

U.S. Pat. No. 6,261,543 discloses perspiration-inhibiting oil-in-water emulsions that contain approximately 6.5 to 10 wt % of an oil and/or fat phase, a mixture of hydrophilic and lipophilic emulsifiers, and 1 wt % of an amphoteric or cationic starch. A corresponding comparison example having a nonionic starch was described as not being shelf-stable at 50° C.

None of these documents deals with the problem of accelerated drying of the roll-on emulsion on the skin.

### BRIEF SUMMARY OF THE INVENTION

An object of the present invention was to make available perspiration-inhibiting oil-in-water emulsions having improved shelf stability, in particular extended shelf stability at temperatures of 40° C. and above. A further object of the present invention was to make available perspiration-inhibiting oil-in-water emulsions having a non-greasy skin feel. A further object of the present invention was to make available perspiration-inhibiting oil-in-water emulsions that dry as quickly as possible on the skin.

A further object of the present invention was to make available cosmetic oil-in-water emulsions having improved shelf stability, in particular extended shelf stability at temperatures of 40° C. and above, for deodorizing, anti-wrinkle, anti-aging, anti-pimple, anti-acne, sebum-regulating, skin-moistening or moisture-donating, light protecting, insect-repelling, self-tanning, or lightening skin treatment. A further object of the present invention was to make available cosmetic oil-in-water emulsions having a non-greasy skin feel, for deodorizing, anti-wrinkle, anti-aging, anti-pimple, anti-acne, sebum-regulating, skin-moistening or moisture-donating, light protecting, insect-repelling, self-tanning, or lightening skin treatment. A further object of the present invention was to make available cosmetic oil-in-water emulsions that dry as quickly as possible on the skin, for deodorizing, anti-wrinkle, anti-aging, anti-pimple, anti-acne, sebum-regulating, skin-moistening or moisture-donating, light protecting, insect-repelling, self-tanning, or lightening skin treatment.

It has been found, surprisingly, that it is possible to manufacture oil-in-water emulsions having a proportion of oil phase or fat phase of at most 6.5 wt %, containing selected oil components in combination with a preferably small proportion of at least one polysaccharide, that are shelf-stable for several weeks even at high temperatures of 45° C. and above and that in addition, after application to the skin, exhibit a drying speed that is perceived by the user as being considerably shortened as compared with the existing art.

A first subject of the present invention is, therefore, a cosmetic oil-in-water emulsion that does not represent a microemulsion and that contains 0.5 to 6.5 wt % oil phase or fat phase, encompassing at least one oil component that is liquid at 20° C., selected from linear and branched saturated mono- or polyvalent C₃ to C₃₀ alkanols that are etherified with at least one propylene oxide unit per molecule, propylene glycol monoesters of branched saturated C₆ to C₃₀ alkanecarboxylic acids and branched saturated C₁₀ to C₃₀ alkanols, at least 60 wt % water, 0.00001 to 38 wt % of at least one cosmetic active substance selected from
perspiration-inhibiting active substances,
deodorizing active substances,
monomers, oligomers, and polymers of amino acids, N—C₂-C₂₄ acylamino acids, the esters and/or the physiologically compatible salts of said substances,
DNA or RNA oligonucleotides,
natural betaine compounds,
vitamins, provitamins, and vitamin precursors of groups A, B, C, E, H, and K, and the esters of the aforesaid substances,
α-hydroxycarboxylic acids, α-ketocarboxylic acids, β-hydroxycarboxylic acids, and their ester, lactone, or salt form,
flavonoids and flavonoid-rich plant extracts,
isoflavonoids and isoflavonoid-rich plant extracts,
polyphenols and polyphenol-rich plant extracts,
ubiquinone and ubiquinol, and derivatives thereof,
silymarin,
ectoin,
repellents,
inorganic and organic UV-filtering substances,
self-tanning active substances,
skin-lightening active substances,
skin-calming active substances,
moisture-donating active substances,
sebum-regulating active substances,
   and at least one polysaccharide, all the quantitative indications being based on the total weight of the emulsion.

The oil-in-water emulsions according to the present invention are notable for containing at least one polysaccharide. Surprisingly, it has been found that the polysaccharide content accelerates drying of the emulsion on the skin. As compared with a polysaccharide-free emulsion, emulsions according to the present invention are perceived by test subjects as drying more quickly on the skin.

### BRIEF DESCRIPTION OF THE SEVERAL VIEWS OF THE DRAWING(S)

Not Applicable

### DETAILED DESCRIPTION OF THE INVENTION

“Polysaccharides” (glycans, polyglycans) is the general term for macromolecular carbohydrates whose molecules are made up of a large number (at least >10, but usually considerably more) monosaccharide molecules (glycoses) glycosidically linked to one another.

Among the polysaccharides preferred according to the present invention are especially the biopolymers starch, cellulose, and dextran, which can be construed as polycondensation products of D-glucose (polyglucosans, glucans), inulin, constituting a polycondensate of D-fructose (polyfructosan, fructan), chitin, and alginic acid.

Both unmodified polysaccharides such as, for example, xanthan or starch, and chemically modified polysaccharide derivatives such as, for example, aluminum starch octenylsuccinate, hydroxypropylmethyl cellulose, or dehydrated xanthan (INCI: Dehydroxanthan Gum), as well as physically modified polysaccharides, for example a starch that has been pre-gelatinized by heat treatment, are understood as polysaccharides suitable according to the present invention. Polysaccharides preferred according to the present invention are selected from starches, in particular from corn, potatoes, and wheat, their constituents such as amylose and amylopectin, starch hydrolysates and starch degradation products such as maltodextrin, the physically or chemically modified starch derivatives, in particular the anionic starch derivatives aluminum starch octenylsuccinate, sodium starch octenylsuccinate, calcium starch octenylsuccinate, distarch phosphates, hydroxyethyl starch phosphates, hydroxypropyl starch phosphates, sodium carboxymethyl starches and sodium starch glycolate, cellulose, the chemically modified cellulose derivatives methyl cellulose, hydroxypropyl cellulose, hydroxyethyl cellulose, hydroxypropylmethyl cellulose, hydroxypropylethyl cellulose, hydroxyethylmethyl cellulose, and carboxymethyl cellulose. Polysaccharides that form gums, such as, for example, guar gum, xanthan gum, dehydroxanthan gum, alginates, in particular sodium alginate, gum arabic, karaya gum, carrageenans, locust bean flour, linseed gums, and agar-agar, can also be contained, but are less preferred. In a particularly preferred embodiment, the compositions according to the present invention are free of polysaccharide gums. In a further particularly preferred embodiment, the compositions according to the present invention are free of guar gum, xanthan gum, dehydroxanthan gum, alginates, in particular sodium alginate, gum arabic, karaya gum, carrageenans, locust bean flour, linseed gums, and agar-agar.

Particularly preferred oil-in-water emulsions according to the present invention are characterized in that the at least one polysaccharide is selected from anionic and nonionic polysaccharides as well as mixtures thereof.

Further particularly preferred oil-in-water emulsions according to the present invention are characterized in that the at least one polysaccharide is selected from anionic and nonionic polysaccharides that do not constitute polysaccharide gums.

Further particularly preferred oil-in-water emulsions according to the present invention are characterized in that the anionic polysaccharide is selected from aluminum starch octenylsuccinate, sodium starch octenylsuccinate, calcium starch octenylsuccinate, distarch phosphates, hydroxyethyl starch phosphates, hydroxypropyl starch phosphates, sodium carboxymethyl starches, sodium starch glycolate, and mixtures thereof. An anionic polysaccharide that is extraordinarily preferred according to the present invention is aluminum starch octenylsuccinate.

Further particularly preferred oil-in-water emulsions according to the present invention are characterized in that the nonionic polysaccharide is selected from starches, starch hydrolysates, cellulose, methyl cellulose, hydroxypropyl cellulose, hydroxyethyl cellulose, hydroxypropylmethyl cellulose, hydroxypropylethyl cellulose, hydroxyethylmethyl cellulose, and mixtures thereof.

Further particularly preferred oil-in-water emulsions according to the present invention are characterized in that at least one polysaccharide is contained in a total quantity from 0.01 to 1.0 wt %, preferably 0.05 to 0.5 wt % and particularly preferably 0.09 to 0.2 wt %, based in each case on the total weight of the emulsion. It was particularly surprising that quicker drying of the emulsion on the skin could be achieved even with relatively small quantities of polysaccharide.

The oil-in-water emulsions according to the present invention are further notable, as compared with the existing art, for a low proportion of an oil phase or fat phase, from 0.5 to 6.5 wt % based on the weight of the entire emulsion. The low proportion of dispersed oil phase or fat phase results in an improved, non-greasy skin feel. In addition, the emulsions according to the present invention represent an outstanding, non-comedogenic basis in particular for cosmetic and dermatologic active substances that are intended for the treatment of greasy, unclean skin and/or acne skin. The emulsions according to the present invention further represent an outstanding basis for sun protection compositions, since it is precisely the fat and emulsifier content that is often responsible for incompatibility reactions of such compositions in response to sunlight. With the emulsions according to the present invention, the risk of incompatibility reactions can be greatly minimized. The emulsions according to the present invention further represent an outstanding basis for self-tanning compositions whose active substances, in particular dihydroxyacetone, are difficult to stabilize in known emulsions because they enter into undefined reactions with numerous emulsion constituents that are usually used, resulting in deactivation of the active substance and discoloration of the cosmetic. An emulsion of this kind additionally offers economic advantages.

Emulsions of this kind usually cannot, however, be manufactured with a viscosity that is necessary for application with a roll-on or ball applicator. One particular challenge of the present invention was, therefore, to manufacture cosmetic oil-in-water emulsions having a proportion of an oil phase or fat phase from 0.5 to 6.5 wt % based on the weight of the entire emulsion, and a viscosity sufficient for application as a roll-on. Included in the oil phase or fat phase, according to the present invention, in addition to the at least one oil component that is liquid at 20° C. (under standard conditions), which is selected from linear and branched saturated mono- or polyvalent C₃ to C₃₀ alkanols that are etherified with at least one propylene oxide unit per molecule, propylene glycol monoesters of branched saturated C₆ to C₃₀ alkanecarboxylic acids, and branched saturated C₁₀ to C₃₀ alkanols, are also fragrances, if present. In addition, fat components that are solid or pasty at 20° C. (under standard conditions) can also be contained. By definition, the emulsifiers are not included in the oil phase or fat phase.

Among the oil components that are liquid at 20° C. (under standard conditions), which are selected from linear and branched saturated mono- or polyvalent C₃ to C₃₀ alkanols that are etherified with at least one propylene oxide per molecule, are preferably propanol, glycerol, propylene glycol, butanol, butanediol, pentanol, decyl alcohol, capryl alcohol, caprylyl alcohol, lauryl alcohol, tridecyl alcohol, myristyl alcohol, palmityl alcohol, cetyl alcohol, stearyl alcohol, arachidyl alcohol, and behenyl alcohol, that are etherified with 1, 2, 3, 4, 5, 6, 7, 8, 9, 10, 11, 12, 13, 14, 15, 16, 17, 18, 19 or 20 propylene oxide units.

Preferred oil-in-water emulsions according to the present invention are characterized in that the oil component i) is selected from addition products of at least six propylene oxide units per molecule to mono- or polyvalent C₃₋₃₀ alkanols, in particular to butanol, butanediol, myristyl alcohol, and stearyl alcohol.

Particularly preferred perspiration-inhibiting oil-in-water emulsions according to the present invention are characterized in that the oil component i) is selected from PPG-3 myristyl ether (obtainable e.g., as a commercial product Witconol® APM), PPG-13 butyl ether, PPG-14 butyl ether (obtainable e.g., as a commercial product Ucon Fluid® AP), PPG-9 butyl ether (obtainable e.g., as a commercial product Breox® B25), PPG-10 butanediol (obtainable e.g., as a commercial product Macol® 57), and PPG-15 stearyl ether (obtainable e.g., as a commercial product Arlamol® E), and mixtures thereof.

Further preferred oil-in-water emulsions according to the present invention are characterized in that the oil component ii) is selected from propylene glycol monoesters of branched saturated C₆ to C₃₀ alkanecarboxylic acids.

Oil components ii) that are particularly preferred according to the present invention are selected from propylene glycol monoisostearate, propylene glycol monoisopalmitate, propylene glycol monoisobehenate, propylene glycol monoisoarachidate, propylene glycol monoisomyristate, propylene glycol monoisocaprate, propylene glycol monoisocaprinate, and propylene glycol monoisocaprylate, and mixtures thereof.

Further preferred oil-in-water emulsions according to the present invention are characterized in that the oil component iii) is selected from branched saturated C₁₀ to C₃₀ alkanols. Oil components iii) that are particularly preferred according to the present invention are selected from isostearyl alcohol, isocetyl alcohol, isomyristyl alcohol, isotridecyl alcohol, isoarachidyl alcohol, isobehenyl alcohol, isocapryl alcohol, isocaprinyl alcohol, isocaprylyl alcohol, and mixtures thereof.

Further preferred oil-in-water emulsions according to the present invention are characterized in that the at least one oil component, selected from the aforementioned groups i), ii), and iii), is contained in a total quantity from 0.1 to 6.5 wt %, preferably 0.3 to 5 wt %, particularly preferably 0.5 to 3 wt %, and extraordinarily preferably 1 to 2 wt %, based in each case on the total weight of the emulsion.

Further preferred oil-in-water emulsions obtainable according to the present invention are characterized in that the oil phase or fat phase is contained in a total quantity from 0.7 to 5 wt %, preferably 1 to 4 wt %, particularly preferably 1.5 to 3 wt %, and extraordinarily preferably 2 to 2.5 wt %, based in each case on the total weight of the emulsion.

It has been found, surprisingly, that the shelf stability of the oil-in-water emulsions according to the present invention can be further increased by the addition of at least one nonionic emulsifier having an HLB value in the range from 3 to 6. Lipophilic emulsifiers of this kind normally stabilize water-in-oil emulsions.

Further preferred oil-in-water emulsions according to the present invention are characterized in that at least one nonionic emulsifier having an HLB value in the range from 3 to 6 is contained.

Nonionic emulsifiers having an HLB value in the range from 3 to 6 that are preferred according to the present invention are selected from linear saturated and unsaturated C₁₂ to C₃₀ alkanols that are etherified with 1 to 4 ethylene oxide units per molecule.

Particularly preferred nonionic emulsifiers having an HLB value in the range from 3 to 6 are selected from Steareth, Ceteth, Myristeth, Laureth, Trideceth, Arachideth, and Beheneth, having respectively 1 to 4 ethylene oxide units per molecule. Steareth-1, Steareth-2, Steareth-3, Ceteth-1, Ceteth-2, Ceteth-3, Myristeth-1, Myristeth-2, Laureth-1, Beheneth-2, Beheneth-3, and Beheneth-4 are extraordinarily preferred, in particular Steareth-2.

Further oil-in-water emulsions that are preferred according to the present invention are characterized in that at least one nonionic emulsifier having an HLB value in the range from 3 to 6 is contained in a total quantity from 1.8 to 3 wt %, preferably 2 to 2.8 wt %, and particularly preferably 2.4 to 2.6 wt %, based in each case on the weight of the entire emulsion.

It has furthermore been found, surprisingly, that the shelf stability of the oil-in-water emulsions according to the present invention can be further increased by the addition of at least one nonionic emulsifier having an HLB value in the range from 12 to 18. Further oil-in-water emulsions preferred according to the present invention are characterized in that at least one nonionic emulsifier having an HLB value in the range from 12 to 18 is contained. Nonionic emulsifiers having an HLB value in the range from 12 to 18 that are preferred according to the present invention are selected from linear saturated and unsaturated C₁₂ to C₂₄ alkanols that are etherified with 7 to 40 ethylene oxide units per molecule. Particularly preferred nonionic emulsifiers having an HLB value in the range from 12 to 18 are selected from Steareth, Ceteth, Myristeth, Laureth, Trideceth, Arachideth, and Beheneth, having respectively 7 to 40 ethylene oxide units per molecule, in particular Steareth-15, Steareth-20, Steareth-21, Arachideth-20, Arachideth-21, Beheneth-20, Beheneth-21, Ceteth-20, Ceteth-30, Ceteth-15, and Myristeth-15.

Further oil-in-water emulsions preferred according to the present invention are characterized in that at least one nonionic emulsifier having an HLB value in the range from 12 to 18 is contained in a total quantity from 1 to 2 wt %, preferably 1.2 to 1.8 wt %, and particularly preferably 1.5 to 1.7 wt %, based in each case on the weight of the entire emulsion.

Further oil-in-water emulsions preferred according to the present invention are characterized in that at least one nonionic emulsifier having an HLB value in the range from 3 to 6, in combination with at least one nonionic emulsifier having an HLB value in the range from 12 to 18, is contained.

Further oil-in-water emulsions preferred according to the present invention are characterized in that Steareth-2 is contained as a nonionic emulsifier having an HLB value in the range from 3 to 6, in combination with Steareth-21 as a nonionic emulsifier having an HLB value in the range from 12 to 18. Emulsions of this kind are notable for particularly favorable shelf and temperature stability.

Further oil-in-water emulsions preferred according to the present invention are characterized in that the weight ratio of the total quantity of nonionic emulsifiers having an HLB value in the range from 3 to 6, and the total quantity of nonionic emulsifiers having an HLB value in the range from 12 to 18, is 0.9 to 3, preferably 1.3 to 1.9.

A variety of oil-in-water emulsifiers and water-in-oil emulsifiers, and their HLB values, are compiled in the table below. These are emulsifiers commonly known to one skilled in the art, such as those listed, for example, in Kirk-Othmer, “Encyclopedia of Chemical Technology,” 3rd ed., 1979, Vol. 8, pp. 913-916. For ethoxylated products, the HLB value is calculated according to the formula HLB=(100-L): 5, where L is the weight proportion of the lipophilic groups, i.e., the fatty alkyl or fatty acyl groups, in the ethylene oxide adducts, expressed as a percentage by weight. The HLB values can be calculated according to Griffin, as presented and tabulated, for example, in the ROMPP Chemie Lexikon [Chemical Lexicon], in particular in the online version of November 2003, and in the handbooks of Fiedler, Kirk-Othmer, and Janistyn cited therein under the keyword “HLB system.” If there are different indications in the literature regarding the HLB value, the HLB value that is closest to the value calculated according to Griffin should be used for the teaching according to the present invention. If an unequivocal HLB value cannot be ascertained in this manner, the HLB value indicated by the manufacturer of the emulsifier is to be used for the teaching according to the present invention. If this is also not possible, the HLB value is to be ascertained experimentally.

| HLB value | Chemical designation |
|---|---|
| 1 | Triglycerides of saturated fatty acids |
| | Glyceryl trioleate |
| 1.5 | Ethylene glycol distearate |
| 1.6 | Pur-Cellin oil |
| 1.8 | Sorbitan trioleate |
| | Glycerol dioleate |
| 2.1 | Sorbitan tristearate |
| 2.4 | Propylene glycol lactostearate |
| 2.7 | Glycerol monooleate |
| | Sorbitol dioleate |
| 2.8 | Glycerol monostearate |
| | Propylene glycol mono-/distearate, not self-emulsifying |
| 2.9 | Ethylene glycol monostearate |
| 3.0 | Decaglycerol decaoleate |
| | Decaglycerol decastearate |
| | Generol 122 (Rapeseed Sterols) |
| | Sucrose distearate |
| 3.1 | Decaglycerol decaoleate |
| | Glyceryl monoricinoleate |
| | Pentaerythrityl monostearate |
| | Pentaerythrityl sesquioleate |
| 3.2 | Ethylene glycol monodistearate, not self-emulsifying |
| | Glycol stearate |
| 3.3 | Glycerol monolaurate |
| 3.4 | Propylene glycol monostearate |
| 3.5 | Ethylene glycol monostearate |
| | Pentaerythrityl monooleate |
| | Polyethylene glycol (100) monooleate |
| 3.6 | Glycerol mono-/dioleate, not self-emulsifying |
| | Monoethoxylauryl ether |
| 3.7 | Sorbitan sesquioleate (Dehymuls SSO) |
| 3.8 | Glycerol monodistearate, not self-emulsifying |
| | Polyethylene glycol (100) monostearate |
| | Diglycerol sesquioleate |
| | N,N-Dimethylcaproamide |
| | Pentaerythritol monotallate |
| | Propylene glycol monolaurate |
| 4.0 | Decaglycerol octaoleate |
| 4.3 | Sorbitan monooleate (Dehymuls SMO) |
| | Diethylene glycol monostearate |
| 4.4 | 1,2-Propylene glycol monodistearate, self-emulsifying |
| 4.5 | Glycerol monostearate palmitate (90%), not self-emulsifying |
| | Propylene glycol monolaurate |
| 4.7 | Sorbitan monostearate (Dehymuls SMS) |
| | Diethylene glycol monooleate |
| 4.8 | Pentaerythritol monolaurate |
| 4.9 | Polyoxyethylene (2) oleyl alcohol (polyoxyethylene (2) oleyl ether) |
| | Polyoxyethylene (2) stearyl alcohol (polyoxyethylene (2) stearyl ether) |
| 5.0 | Ethylene glycol monodistearate |
| | Generol 122 E 5 (PEG-5 Soy Sterol) |
| | Polyethylene glycol (100) monoricinoleate |
| | Polyethylene glycol (200) distearate |
| | Polyglyceryl-3-isostearate (e.g., Isolan GI 34 of Tego) |
| 5.9 | Polyethylene glycol (200) dilaurate |
| 6.0 | Decaglycerol tetraoleate |
| | Polyethylene glycol (100) monolaurate |
| | Polyethylene glycol (200) dioleate |
| 6.1 | Diethylene glycol monolaurate (diglycol laurate) |
| 6.3 | Polyethylene glycol (300) dilaurate |
| 6.4 | Glycerol monoricinoleate |
| | Glycerol sorbitan monolaurate |
| 6.5 | Diethylene glycol monolaurate |
| | Sodium stearoyl 2-lactylate |
| 6.7 | Sorbitan monopalmitate |
| 6.8 | Glycerol monococoate |
| | Glycerol monolaurate |
| 7.0 | Polyoxyethylene (2) C₁₀-C₁₄ fatty alcohol ether, Laureth-2 (Dehydol LS 2) |
| | Sucrose distearate |
| 7.2 | Polyethylene glycol (400) dioleate |
| | Sucrose dioleate |
| 7.4 | Polyethylene glycol (100) monolaurate |
| | Sucrose dipalmitate |
| 7.5 | Sucrose dipalmitate |
| 7.6 | Glycerol sorbitan laurate |
| 7.8 | Polyethylene glycol (400) distearate |
| 7.9 | Polyethylene glycol (200) monostearate |
| | Polyoxyethylene (3) tridecyl alcohol |
| 8-8.2 | Polyethylene glycol (400) distearate |
| 8.0 | Polyoxyethylene (3) C₁₀-C₁₄ fatty alcohol ether, Laureth-3 (Dehydol LS 3) |
| | N,N-Dimethyl lauramide |
| | Sodium lauroyl lactylate, sodium lauroyl 2-lactylate |
| | Polyethylene glycol (200) monooleate |
| | Polyethylene glycol (220) monotallate |
| | Polyethylene glycol (1500) dioleate |
| | Polyoxyethylene (4) oleyl alcohol |
| | Polyoxyethylene (4) stearylcetyl ether |
| 8-8.2 | Triglycerol monooleate |
| 8.3 | Diethylene glycol monolaurate |
| 8.4 | Polyoxyethylene (4) cetyl ether |
| | Polyoxyethylene glycol (400) dioleate |
| 8.5 | Sodium caproyl lactylate |
| | Polyethylene glycol (200) monostearate |
| | Sorbitan monooleate |
| 8.6 | Sorbitan monolaurate (Dehymuls SML) |
| | Polyethylene glycol (200) monolaurate |
| 8.8 | Polyoxyethylene (4) myristyl ether |
| | Polyethylene glycol (400) dioleate |
| 8.9 | Nonylphenol, polyoxyethylated with 4 mol EO |
| 9.0 | Oleth-5 (e.g., Eumulgin O 5) |
| 9.2-9.7 | Polyoxyethylene (4) lauryl alcohol (depending on commercial product, e.g., Brij 30, Dehydol LS |
| | 4) |
| 9.3 | Polyoxyethylene (4) tridecyl alcohol |
| 9.6 | Polyoxyethylene (4) sorbitan monostearate |
| 9.8 | Polyethylene glycol (200) monolaurate |
| 10-11 | Polyethylene glycol (400) monooleate |
| 10.0 | Didodecyldimethylammonium chloride |
| 10.0 | Polyethylene glycol (200) monolaurate |
| | Polyethylene glycol (400) dilaurate |
| | Polyethylene glycol (600) dioleate |
| | Polyoxyethylene (4) sorbitan monostearate |
| | Polyoxyethylene (5) sorbitan monooleate |
| 10.2 | Polyoxyethylene (40) sorbitol hexaoleate |
| 10.4-10.6 | Polyoxyethylene glycol (600) distearate |
| 10.5 | Polyoxyethylene (20) sorbitan tristearate |
| 10.6 | Sucrose monostearate |
| 10.7 | Sucrose monooleate |
| 11-11.4 | Polyethylene glycol (400) monooleate |
| 11.0 | Polyethylene glycol (350) monostearate |
| | Polyethylene glycol (400) monotallate |
| | Polyoxyethylene glycol (7) monostearate |
| | Polyoxyethylene glycol (8) monooleate |
| | Polyoxyethylene (20) sorbitan trioleate |
| | Polyoxyethylene (6) tridecyl alcohol |
| 11.1 | Polyethylene glycol (400) monostearate |
| 11.2 | Polyoxyethylene (9) monostearate |
| | Sucrose monooleate |
| | Sucrose monostearate |
| 11.4 | Polyoxyethylene (50) sorbitol hexaoleate |
| | Sucrose monotallate |
| | Sucrose stearate palmitate |
| 11.6 | Polyoxyethylene glycol (400) monoricinoleate |
| 11.7 | Sucrose monomyristate |
| | Sucrose monopalmitate |
| 12.0 | PEG-10 Soy Sterol (e.g., Generol 122 E 10) |
| | Triethanolamine oleate |
| 12.2-12.3 | Nonylphenol, ethoxylated with 8 mol EO |
| 12.2 | Sucrose monomyristate |
| 12.4 | Sucrose monolaurate |
| | Polyoxyethylene (10) oleyl alcohol, polyoxyethylene (10) oleyl ether |
| | Polyoxyethylene (10) stearyl alcohol, polyoxyethylene (10) stearyl ether |
| 12.5 | Polyoxyethylene (10) stearyl cetyl ether |
| 12.7 | Polyoxyethylene (8) tridecyl alcohol |
| 12.8 | Polyoxyethylene glycol (400) monolaurate |
| | Sucrose monococoate |
| 12.9 | Polyoxyethylene (10) cetyl ether |
| 13 | Glycerol monostearate, ethoxylated (20 mol EO) |
| 13.0 | Eumulgin O 10 (Polyoxyethylene (10) oleyl ether) |
| | Eumulgin 286 (Nonoxynol-10) |
| | Eumulgin B 1 (Ceteareth-12) |
| 13.0 | C12 fatty amines, ethoxylated (5 mol EO) |
| 13.1 | Nonylphenol, ethoxylated (9.5 mol EO) |
| 13.2 | Polyethylene glycol (600) monostearate |
| | Polyoxyethylene (16) tall oil |
| 13.3 | Polyoxyethylene (4) sorbitan monolaurate |
| 13.5 | Nonylphenol, ethoxylated (10.5 mol EO) |
| | Polyethylene glycol (600) monooleate |
| 13.7 | Polyoxyethylene (10) tridecyl alcohol |
| | Polyethylene glycol (660) monotallate |
| | Polyethylene glycol (1500) monostearate |
| | Polyoxyethylene glycol (1500) dioleate |
| 13.9 | Polyethylene glycol (400) monococoate |
| | Polyoxyethylene (9) monolaurate |
| 14-16 | Eumulgin HRE 40 (castor oil, ethoxylated with 40 EO and hydrogenated) |
| 14.0 | Polyoxyethylene (12) lauryl ether |
| | Polyoxyethylene (12) tridecyl alcohol |
| 14.2 | Polyoxyethylene (15) stearyl alcohol |
| 14.3 | Polyoxyethylene (15) stearylcetyl ether |
| 14.4 | Mixture of C12-C15 fatty alcohols with 12 mol EO |
| 14.5 | Polyoxyethylene (12) lauryl alcohol |
| 14.8 | Polyoxyethylene glycol (600) monolaurate |
| 14.9-15.2 | Sorbitan monostearate, ethoxylated with 20 EO (e.g., Eumulgin SMS 20) |
| 15-15.9 | Sorbitan monooleate, ethoxylated with 20 EO (e.g., Eumulgin SMO 20) |
| 15.0 | PEG-20 glyceryl stearate (e.g., Cutina E 24) |
| | PEG-40 Castor Oil (e.g., Eumulgin RO 40) |
| | Decyl glucoside (Oramix NS 10) |
| | Dodecyl glucoside (Plantaren APG 600) |
| | Dodecyltrimethylammonium chloride |
| | Nonylphenol, ethoxylated with 15 mol EO |
| | Polyethylene glycol (1,000) monostearate |
| | Polyoxyethylene (600) monooleate |
| 15-17 | Eumulgin HRE 60 (castor oil, ethoxylated with 60 EO and hydrogenated) |
| 15.3 | C12 fatty amines, polyethoxylated with 12 mol EO |
| | Polyoxyethylene (20) oleyl alcohol, polyoxyethylene (20) oleyl ether |
| 15.4 | Polyoxyethylene (20) stearyl cetyl ether (e.g., Eumulgin B 2 (Ceteareth-20)) |
| 15.5 | Polyoxyethylene (20) stearyl alcohol |
| 15.6 | Polyoxyethylene glycol (1,000) monostearate |
| | Polyoxyethylene (20) sorbitan monopalmitate |
| 15.7 | Polyoxyethylene (20) cetyl ether |
| 15.9 | Disodium triethanolamine distearyl heptaglycol ether sulfosuccinate |
| 16.0 | Nonylphenol ethoxylated with 20 mol EO |
| | Polyoxyethylene (25) propylene glycol stearate |
| 16-16.8 | Polyoxyethylene (30) monostearate |
| 16.3-16.9 | Polyoxyethylene (40) monostearate |
| 16.5-16.7 | Polyoxyethylene (20) sorbitan monolaurate (e.g., Eumulgin SML 20) |
| 16.6 | Polyoxyethylene (20) sorbitol |
| 16.7 | C18 fatty amines, polyethoxylated with 5 mol EO |
| | Polyoxyethylene (23) lauryl alcohol |
| 17.0 | Ceteareth-30, e.g., Eumulgin B 3 |
| | Octyl glucoside (Triton CG 110) |
| | Polyoxyethylene (30) glyceryl monolaurate |
| 17.1 | Nonylphenol, ethoxylated with 30 mol EO |
| 17.4 | Polyoxyethylene (40) stearyl alcohol |

Further oil-in-water emulsions particularly preferred according to the present invention are characterized in that Steareth-2, Steareth-21, and PPG-15 stearyl ether are contained. Emulsions of this kind are notable for particularly high shelf and temperature stability, and at the same time contribute to an improved, non-sticky skin feel.

Oil-in-water emulsions that are extraordinarily preferred according to the present invention are characterized in that in addition to Steareth-2, Steareth-21, and PPG-15 stearyl ether, aluminum starch octenylsuccinate is contained as a polysaccharide. This polysaccharide is obtainable, for example, from National Starch under the commercial names Dry Flo and Dry Flo Plus. Further oil-in-water emulsions extraordinarily preferred according to the present invention are characterized in that in addition to Steareth-2, Steareth-21, and PPG-15 stearyl ether, at least one distarch phosphate is contained. This polysaccharide is obtainable, for example, from Agrana under the commercial name Maize PO 4 PH “B”. Emulsions of this kind are notable for particularly high shelf and temperature stability, an outstanding, non-sticky skin feel, and optimum drying properties.

The proportion of water in the composition according to the present invention is at least 60 wt %, preferably 65 to 90 wt %, particularly preferably 70 to 85 wt %, extraordinarily preferably 75 to 80 wt %, based in each case on the entire composition.

Further oil-in-water emulsions preferred according to the present invention are characterized in that a total of at most 3 wt %, preferably at most 1 wt %, and particularly preferably 0 wt %, based in each case on the weight of the entire emulsion, of monovalent C₁ to C₃ alkanols, such as ethanol or isopropanol, is contained. Under certain conditions, the emulsions according to the present invention can be destabilized in terms of their shelf and/or temperature stability by an addition of ethanol or isopropanol, especially in larger quantities (e.g., 5 wt % and more).

The oil-in-water emulsions according to the present invention were developed in particular for roll-on products, i.e., for application with a ball applicator or roll-on applicator. For optimum metering properties, the emulsion must have neither too low nor too high a viscosity. Oil-in-water emulsions preferred according to the present invention are, therefore, characterized by a viscosity in the range from 1,000 to 5,000 mPas, preferably 1,500 to 4,000 mPas, and particularly preferably 1,700 to 2,200 mPas. These viscosity indications refer to measurements with a Brookfield viscosimeter, which were carried out one day after manufacture of the emulsion, using an RV 4 spindle at a shear rate (spindle rotation speed) of 20 s⁻¹ without Helipath, at an ambient temperature and sample temperature of 20° C. in each case.

### Perspiration-Inhibiting Active Substances.

Oil-in-water emulsions preferred according to the present invention are characterized in that the cosmetic active substance c) is selected from perspiration-inhibiting active substances. Perspiration-inhibiting or antiperspirant active substances preferred according to the present invention are selected from the water-soluble astringent inorganic and organic salts of aluminum, zirconium, and zinc, and any mixtures of said salts. Particularly preferred antiperspirant active substances are selected from the aluminum chlorohydrates, in particular the aluminum chlorohydrates having the general formula [Al₂(OH)₅Cl.2-3H₂O]ₙ, which can be present in non-activated or in activated (depolymerized) form, also aluminum sesquichlorohydrate, aluminum chlorohydrex propylene glycol (PG) or polyethylene glycol (PEG), aluminum sesquichlorohydrex PG or PEG, aluminum PG dichlorohydrex or aluminum PEG dichlorohydrex, aluminum hydroxide, further selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum zirconium chlorohydrate-glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAI(SO₄)₂.12H₂O, alum), aluminum undecylenoyl collagen amino acid, sodium aluminum lactate+aluminum sulfate, sodium aluminum chlorohydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxyallantoinate, sodium aluminum chlorohydroxylactate, zinc chloride, zinc sulfocarbolate, zinc sulfate, and zirconium chlorohydrate. According to the present invention, “water solubility” means a solubility of at least 5 wt % at 20° C. “5 wt %” means that 5 g of the antiperspirant active substance is soluble in 95 g of water at 20° C. The antiperspirant active substances are by preference used according to the present invention as aqueous solutions. When zirconium salts and aluminum-zirconium salts are used, care must be taken that the prefabricated aqueous active substance solutions are prepared as freshly as possible. With extended storage time, the zirconium compounds can tend to polymerize, which is associated with both a loss of activity and an increase in viscosity.

Particularly preferred perspiration-inhibiting emulsions according to the present invention are characterized in that the at least one antiperspirant active substance is contained in a quantity from 1 to 38 wt %, by preference 5 to 25 wt %, and in particular 10 to 20 wt %, based in each case on the total weight of the active substance in the entire composition. In a particularly preferred embodiment, the composition contains an astringent aluminum salt, in particular aluminum chlorohydrate, that is marketed e.g., in the form of an aqueous solution as Locron® L by Clariant, as Chlorhydrol® and in activated form as Reach® 501 by Reheis. Under the designation Reach®301, Reheis offers an aluminum sesquichlorohydrate that is also particularly preferred. The use of aluminum zirconium tetrachlorohydrex glycine complexes, which are commercially available e.g., from Reheis under the designation Rezal® 36G, can also be particularly preferred according to the present invention.

### Deodorizing Active Substances.

Further oil-in-water emulsions preferred according to the present invention are characterized in that the cosmetic active substance c) is selected from deodorizing substances. Deodorizing active substances preferred according to the present invention are odor absorbers, ion exchangers acting in deodorizing fashion, germ-inhibiting agents, prebiotically active components, and enzyme-inhibitors or, particularly preferably, combinations of the aforesaid active substances.

Silicates serve as odor absorbers that also, simultaneously, advantageously assist the rheological properties of the composition according to the present invention. Among the silicates that are particularly advantageous according to the present invention are principally sheet silicates, and among them in particular montmorillonite, kaolinite, illite, beidellite, nontronite, saponite, hectorite, bentonite, smectite, and talc. Further advantageous odor absorbers are, for example, zeolites, zinc ricinoleate, cyclodextrins, certain metal oxides such as, for example, aluminum oxide, and chlorophyll. They are used preferably in a quantity from 0.1 to 10 wt %, particularly preferably 0.5 to 7 wt %, and extraordinarily preferably 1 to 5 wt %, based in each case on the entire composition.

Germ-inhibiting or antimicrobial active substances that are preferred according to the present invention are, in particular, organohalogen compounds and organohalides, quaternary ammonium compounds, a number of plant extracts, and zinc compounds. These include, among others, triclosan, chlorhexidine and chlorhexidine gluconate, 3,4,4′-trichlorocarbanilide, bromochlorophen, dichlorophen, chlorothymol, chloroxylenol, hexachlorophene, dichloro-m-xylenol, dequalinium chloride, domiphen bromide, ammonium phenolsulfonate, benzalkonium halides, benzalkonium cetyl phosphate, benzalkonium saccharinate, benzethonium chloride, cetylpyridinium chloride, laurylpyridinium chloride, laurylisoquinolinium bromide, methylbenzedonium chloride. Also preferred as germ-inhibiting active substances are phenol, phenoxyethanol, zinc lactate, disodium dihydroxyethylsulfosuccinyl undecylenate, sodium bicarbonate, sodium phenolsulfonate and zinc phenolsulfonate, ketoglutaric acid, terpene alcohols such as, for example, farnesol, chlorophyll-copper complexes, α-monoalkylglycerol ethers having a branched or linear, saturated or unsaturated, optionally hydroxylated C₆ to C₂₂ alkyl radical, particularly preferably α-(2-ethylhexyl)glycerol ether, obtainable commercially as Sensiva® SC 50 (ex Schülke & Mayr), carboxylic acid esters of mono-, di- and triglycerol (e.g., glycerol monolaurate, diglycerol monocaprinate), lantbiotics, and plant extracts (e.g., green tea and constituents of linden blossom oil).

Further preferred deodorant active substances are selected from prebiotically active components, among which are to be understood, according to the present invention, those components that inhibit only, or at least predominantly, the odor-forming germs of the skin microflora, but not the desirable (i.e., non-odor-forming) germs that form part of a healthy skin microflora. Explicitly encompassed here are the active substances disclosed in German Applications DE 10333245 and DE 10 2004 011 968 as prebiotically active; these include conifer extracts, in particular from the group of the Pinaceae, and plant extracts from the group of the Sapindaceae, Araliaceae, Lamiaceae, and Saxifragaceae, in particular extracts from Picea spp., Paullinia sp., Panax sp., Lamium album, or Ribes nigrum, and mixtures of said substances.

Further preferred deodorant active substances are obtainable from the perfume oils having a germ-inhibiting action, and from the Deosafe perfume oils obtainable from the Symrise company, formerly Haarmann+Reimer.

Among the enzyme inhibitors for purposes of the present invention are substances that inhibit the enzymes responsible for perspiration breakdown, in particular arylsulfatase, β-glucuronidase, aminoacylase, esterases, lipases, and/or lipoxigenases, e.g., preferably trialkylcitric acid esters, in particular triethyl citrate, or zinc glycinate. Further substances inhibiting the enzymes and germs responsible for perspiration breakdown, for example arylsulfatase, β-glucuronidase, aminoacylase, esterases, lipases, and/or lipoxigenases, are disclosed in WO 03/039505 A2, WO 01/99376 A2, EP 1430879 A2, EP 1428520 A2, EP 1738803 A1, EP 1576946 A1, and DE 10216368 A1.

Further emulsions preferred according to the present invention are characterized in that at least one deodorizing active substance is contained in a quantity from 0.1 to 10 wt %, preferably 0.2 to 7 wt %, particularly preferably 0.3 to 5 wt %, and extraordinarily preferably 0.4 to 1.0 wt %, based in each case on the total weight of the active substance in the entire composition.

Further emulsions preferred according to the present invention are characterized in that that least one cosmetic active substance c), selected from monomers, oligomers, or polymers of amino acids, N—C₂-C₂₄ acylamino acids, and/or the esters and/or physiologically compatible salts of said substances, is contained. Many of these active substances are used as anti-aging active substances and/or have a favorable effect on the moisture budget of the skin and/or have a skin-calming action.

The monomers of the amino acids and/or of the N—C₂-C₂₄ acylamino acids are preferably selected from alanine, arginine, asparagine, aspartic acid, canavanine, citrulline, cysteine, cystine, dipalmitoyl hydroxyproline, desmosine, glutamine, glutamic acid, glycine, histidine, homophenylalanine, hydroxylysine, hydroxyproline, isodesmosine, isoleucine, leucine, lysine, methionine, methylnorleucine, ornithine, phenylalanine, proline, pyroglutamic acid, sarcosine, serine, taurine, threonine, thyroxine, tryptophan, tyrosine, valine, N-acetyl-L-cysteine, zinc pyroglutamate, sodium octanoyl glutamate, sodium decanoyl glutamate, sodium lauroyl glutamate, sodium myristoyl glutamate, sodium cetoyl glutamate, and sodium stearoyl glutamate. Lysine, serine, zinc pyroglutamate and sodium pyroglutamate, and sodium lauroyl glutamate are particularly preferred.

The C₂ to C₂₄ acyl radical with which the amino acids, in particular the aforesaid preferred amino acids, are derivatized on the amino group is preferably selected from an acetyl, propanoyl, butanoyl, pentanoyl, hexanoyl, heptanoyl, octanoyl, nonanoyl, decanoyl, undecanoyl, lauroyl, tridecanoyl, myristoyl, pentadecanoyl, cetoyl, palmitoyl, stearoyl, elaidoyl, arachidoyl, or behenoyl radical. Mixtures of C₈ to C₁₈ acyl radicals are also referred to as cocoyl radicals, and are likewise preferred substituents.

With the aforementioned C₂ to C₂₄ radicals, the amino acids that carry an OH group can also be esterified at that OH group. An example of this that is preferred according to the present invention is hydroxyproline that is N-acylated and esterified with two, preferably linear, C₂ to C₂₂ fatty acid esters, particularly preferably dipalmitoyl hydroxyproline, which is obtainable e.g., under the designation Sepilift DPHP from the Seppic company.

The physiologically compatible salts of the amino acids or amino acid derivatives preferred according to the present invention are selected from the ammonium, alkali-metal, magnesium, calcium, aluminum, zinc, and manganese salts. The sodium, potassium, magnesium, aluminum, zinc, and manganese salts are particularly preferred.

“Amino acid oligomers” are understood according to the present invention as peptides having 2 to 30, preferably 2 to 15, amino acids. The oligomers of the amino acids and/or of the N—C₂-C₂₄ acylamino acids are preferably selected from di-, tri-, tetra-, penta-, hexa-, or pentadecapeptides, which can be acylated and/or esterified. Many of these amino acid oligomers stimulate collagen synthesis or are capable of recruiting cells of the immune system such as mast cells and macrophages, which then, via the release of growth factors, induce repair processes in the tissue, e.g., collagen synthesis, or are capable of binding to the Arg-Phe-Lys sequence in thrombospondin I (TSP-1) and thereby releasing active TGF-β (tissue growth factor), which induces the synthesis of collagen in dermal fibroblasts. Amino acid oligomers of this kind are preferably used as active substances against skin aging.

Optionally N-acylated and/or esterified dipeptides preferred according to the present invention are acetyl-citrullyl-arginine (e.g., Exsy-Algine of Exsymol having the INCI name Acetyl Citrull Amido Arginine), Tyr-Arg (Dipeptide-1), Val-Trp (Dipeptide-2), Asn-Phe, Asp-Phe, N-palmitoyl-β-Ala-His, N-acetyl-Tyr-Arg-hexyldecyl ester (e.g., Calmosensine of Sederma), carnosine (β-Ala-His), and N-palmitoyl-Pro-Arg. Optionally N-acylated and/or esterified tripeptides preferred according to the present invention are Gly-His-Lys, which is obtainable e.g., under the designation “Omega CH Activator” from the GfN company or in acylated form (N-palmitoyl-Gly-His-Lys) under the designation Biopeptide CL from Sederma, but also represents (in acylated form) a constituent of the Matrixyl 3000 product of Sederma. The tripeptide Gly-His-Lys can also be used as a copper salt (Cu²⁺), and can be obtained as such from ProCyte Corporation. Analogs of Gly-His-Lys can also be used, a maximum of two amino acids being substituted with other suitable amino acids. According to the present invention, Ala, Leu, and Ile are suitable for substituting Gly. The amino acids preferred according to the present invention that can replace His or Lys contain a side chain having a nitrogen atom that is predominantly present in charged fashion at pH 6, e.g., Pro. Lys, Arg, His, desmosine, and isodesmosine. Particularly preferably, Lys is replaced by Arg, Orn, or citrulline. A further tripeptide preferred according to the present invention is Gly-His-Arg (INCI name: Tripeptide-3) and its derivative N-myristoyl-Gly-His-Arg which is obtainable, for example, under the designation Collasyn 314-GR from Therapeutic Peptide Inc.; further tripeptides preferred according to the present invention are selected from Lys-Val-Lys, Lys-Val-Dab (Dab=diaminobutyric acid), Lys-Phe-Lys, Lys-Ile-Lys, Dab-Val-Lys, Lys-Val-Orn, Lys-Val-Dap (Dap=diaminopropionic acid), Dap-Val-Lys, palmitoyl-Lys-Val-Lys, obtainable e.g., from the Pentapharm company under the designation SYN®-COLL, Lys-Pro-Val, Tyr-Tyr-Val, Tyr-Val-Tyr, Val-Tyr-Val (Tripeptide-2), Tripeptide-4 (e.g., ATPeptide, to be obtained via IMPAG), His-Ala-Orn N-elaidoyl-Lys-Phe-Lys, and N-acetyl-Arg-Lys-Arg-NH₂.

Optionally N-acylated and/or esterified tetrapeptides preferred according to the present invention are selected from rigin and rigin-based tetrapeptides as well as ALAMCAT tetrapeptides. Rigin has the sequence Gly-Gln-Pro-Arg. Rigin-based tetrapeptides encompass the rigin analogs and rigin derivatives, in particular the N-palmitoyl-Gly-Gln-Pro-Arg, particularly preferred according to the present invention, that is obtainable e.g., under the designation Eyeliss from Sederma, but also represents a constituent of the Matrixyl 3000 product of Sederma. Among the rigin analogs are those in which the four amino acids are rearranged and/or in which a maximum of two amino acids are substituted as compared with rigin, e.g., the sequence Ala-Gln-Thr-Arg. Preferably, at least one of the amino acids of the sequence has a Pro or Arg, and particularly preferably, the tetrapeptide contains both Pro and Arg, such that their sequence and position can vary. The substituting amino acids can be selected from any amino acid defined below. Particularly preferred rigin-based tetrapeptides encompass: Xaa-Xbb-Arg-Xcc, Xaa-Xbb-Xcc-Pro, Xaa-Xbb-Pro-Arg, Xaa-Xbb-Pro-Xcc, Xaa-Xbb-Xcc-Arg, where Xaa, Xbb and Xcc can be amino acids identical to or different from one another, and Xaa is selected from Gly and from the amino acids that can substitute for Gly, Xbb is selected from Gln and from the amino acids that can substitute for Gln, Xcc is selected from Pro or Arg and from the amino acids that can substitute for Pro and Arg.

The preferred amino acids that can replace Gly contain an aliphatic side chain, e.g., β-Ala, Ala, Val, Leu, Pro, sarcosine (Sar) and isoleucine (Me).

The preferred amino acids that can replace Gln contain a side chain having an amino group that is present in predominantly uncharged fashion at neutral pH (pH 6-7), e.g., Asn, Lys, Orn, 5-hydroxyproline, citrulline, and canavanine.

The preferred amino acids that can replace Arg contain a side chain having a nitrogen atom that is present in predominantly charged fashion at pH 6, e.g., Pro, Lys, His, desmosine, and isodesmosine.

According to the present invention, Gly-Gln-Arg-Pro and Val-Val-Arg-Pro are preferred as rigin analogs.

ALAMCAT tetrapeptides are tetrapeptides that contain at least one amino acid having an aliphatic side chain, e.g., β-Ala, Alan, Val, Leu, Pro, sarcosine (Sar), and isoleucine (Ile). ALAMCAT tetrapeptides furthermore contain at least one amino acid having a side chain with an amino group that is present in predominantly uncharged fashion at neutral pH (pH 6-7), e.g., Gln, Asn, Lys, Orn, 5-hydroxyproline, citrulline, and canavanine. ALAMCAT tetrapeptides furthermore contain at least one amino acid having a side chain with a nitrogen atom that is present in predominantly charged fashion at pH 6, e.g., Arg, Pro, Lys, His, desmosine, and isodesmosine. ALAMCAT tetrapeptides can contain any desired amino acid as a fourth amino acid; preferably, however, the fourth amino acid is also selected from the three groups recited above.

Optionally N-acylated and/or esterified pentapeptides preferred according to the present invention are selected from Lys-Thr-Thr-Lys-Ser and its N-acylated derivatives, particularly preferably N-palmitoyl-Lys-Thr-Thr-Lys-Ser, which is obtainable from the Sederma company under the designation Matrixyl, furthermore N-palmitoyl-Tyr-Gly-Gly-Phe-Met, Val-Val-Arg-Pro-Pro, N-palmitoyl-Tyr-Gly-Gly-Phe-Leu, Gly-Pro-Phe-Pro-Leu, and N-benzyloxycarbonyl-Gly-Pro-Phe-Pro-Leu (the latter two represent serine proteinase inhibitors to inhibit desquamation). Optionally N-acylated and/or esterified hexapeptides preferred according to the present invention are Val-Gly-Val-Ala-Pro-Gly and its N-acylated derivatives, particularly preferably N-palmitoyl-Val-Gly-Val-Ala-Pro-Gly, which is obtainable from the Sederma company under the designation Biopeptide EL, furthermore Acetyl Hexapeptide-3 (Argireline of Lipotec), Hexapeptide-4 (e.g., Collasyn 6KS of Therapeutic Peptide Inc. (TPI)), Hexapeptide-5 (e.g., Collasyn 6VY of TPI), Myristoyl Hexapeptide-5 (e.g., Collasyn 614VY of TPI), Myristoyl Hexapeptide-6 (e.g., Collasyn 614VG of TPI), Hexapeptide-8 (e.g., Collasyn 6KS of TPI), Myristoyl Hexapeptide-8 (e.g., Collasyn Lipo-6KS of TPI), Hexapeptide-9 (e.g., Collaxyl of Vincience), and Hexapeptide-10 (e.g., Collaxyl of Vincience or Seriseline of Lipotec), Ala-Arg-His-Leu-Phe-Trp (Hexapeptide-1), Acetyl Hexapeptide-1 (e.g., Modulene of Vincience), Acetyl Glutamyl Hexapeptide-1 (e.g., SNAP-7 of Centerchem), Hexapeptide-2 (e.g., Melanostatine-DM of Vincience), Ala-Asp-Leu-Lys-Pro-Thr (Hexapeptide-3, e.g., Peptide 02 of Vincience), Val-Val-Arg-Pro-Pro-Pro, Hexapeptide-4 (e.g., Collasyn 6KS of Therapeutic Peptide Inc. (TPI)), Hexapeptide-5 (e.g., Collasyn 6VY of TPI), Myristoyl Hexapeptide-5 (e.g., Collasyn 614VY of TPI), Myristoyl Hexapeptide-6 (e.g., Collasyn 614VG of TPI), Ala-Arg-His-methylnorleucine-homophenylalanine-Trp (Hexapeptide-7), Hexapeptide-8 (e.g., Collasyn 6KS of TPI), Myristoyl Hexapeptide-8 (e.g., Collasyn Lipo-6KS of TPI), Hexapeptide-9 (e.g., Collaxyl of Vincience), Hexapeptide-10 (e.g., Collaxyl of Vincience or Seriseline of Lipotec) and Hexapeptide-11 (e.g., Peptamide-6 of Arch Personal Care). A pentadecapeptide preferred according to the present invention is, for example, the raw material Vinci 01 of Vincience (Pentadecapeptide-1). A further preferred amino acid oligomer is the peptide derivative L-glutamylaminoethyl indole (Glistin of Exsymol).

Particularly preferred according to the present invention is the combination of N-palmitoyl-Gly-His-Lys and N-palmitoyl-Gly-Gln-Pro-Arg, as obtainable, for example, in the raw material Matrixyl 3000 of the Sederma company.

The polymers of the amino acids and/or of the N—C₂-C₂₄ acylamino acids are preferably selected from vegetable and animal protein hydrolysates and/or proteins. Animal protein hydrolysates are, for example, elastin, collagen, keratin, silk, and milk protein hydrolysates, which can also be present in the form of salts. Vegetable protein hydrolysates, for example, soy, wheat, almond, pea, potato, and rice protein hydrolysates, are preferred according to the present invention. Corresponding commercial products are, for example, DiaMin® (Diamalt), Gluadin® (Cognis), Lexein® (Inolex), and Crotein® (Croda). Soy protein hydrolysates are particularly preferred, e.g., the commercial products Phytokine of Coletica or Ridulisse C of Silab. Protein hydrolysates also contain monomeric amino acids and oligopeptides; their composition is normally not defined.

It is also possible to use acyl derivatives of the protein hydrolysates, e.g., in the form of their fatty acid condensation products. Corresponding commercial products are, for example, Lamepon® (Cognis), Gluadin® (Cognis), Lexein® (Inolex), Crolastin® or Crotein® (Croda).

Cationized protein hydrolysates are also preferred according to the present invention. Cationic protein hydrolysates whose underlying protein component has a molecular weight from 100 to 25,000 dalton, preferably 250 to 5,000 dalton, are particularly preferred. Quaternized amino acids and mixtures thereof are also to be understood as cationic protein hydrolysates. The cationic protein hydrolysates can moreover also be further derivatized. Some of the products recited under INCI names in the “International Cosmetic Ingredient Dictionary and Handbook,” (seventh edition 1997, The Cosmetic, Toiletry, and Fragrance Association, Washington, D.C.) and available commercially may be listed as typical examples of cationic protein hydrolysates and derivatives used according to the present invention: Cocodimonium Hydroxypropyl Hydrolyzed Collagen, Steardimonium Hydroxypropyl Hydrolyzed Collagen, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl. The plant-based cationic protein hydrolysates and derivatives are extraordinarily preferred.

In a further preferred embodiment, the polymers of the amino acids are selected from DNA repair enzymes.

DNA repair enzymes preferred according to the present invention are photolyase and T4 endonuclease V, the latter hereinafter abbreviated “T4N5”. These two enzymes are already known in the existing art as DNA repair enzymes. “DNA repair” is to be understood, by definition, as the cleavage or removal of UV-induced pyrimidine dimers from DNA.

“Photolyase” is the abbreviation for deoxyribopyrimidine photolyase or DNA photolyase, an enzyme having the classification number EC 4.1.99.3. A particularly efficient photolyase derives from Anacystis nidulans, a phototrophic marine microorganism. The photolyase from A. nidulans is now obtained in industrially relevant quantities from E. coli. Photolyase is dependent on light for activation.

The enzyme T4 Endonuclease V is produced by the denV gene of the T4 bacteriophage, and is one of the phosphodiesterases that hydrolytically cleave nucleic acids at the (5′-3′) bond. T4N5 is active even without the influence of light.

The use of liposome-encapsulated DNA repair enzymes is particularly preferred according to the present invention. Liposome-encapsulated photolyase is obtainable commercially, for example, under the product designation Photosome™, and liposome-encapsulated T4N5, for example, under the designation Ultrasome™, from the AGI Dermatics company, USA.

Preferred emulsions according to the present invention are characterized in that they contain at least one of the raw materials Photosome™ or Ultrasome™ in a total quantity from 0.1 to 10 wt %, preferably 0.5 to 5.0 wt %, and particularly preferably 1.0 to 4.0 wt %, based in each case on the entire emulsion.

Preferred emulsions according to the present invention are characterized in that they contain at least one monomer, oligomer, or polymer of amino acids, N—C₂-C₂₄ acylamino acids, and/or the esters and/or physiologically compatible salts of said substances in a total quantity from 0.01 to 10 wt %, preferably 0.1 to 5 wt %, and particularly preferably 0.1 to 3 wt %, based in each case on the entire emulsion.

In a further preferred embodiment, the monomers, oligomers, and polymers of amino acids, N—C₂-C₂₄ acylamino acids, esters and/or physiologically compatible salts of said substances are present in carrierized form, in particular applied onto finely particulate powdered substrates such as silica gel, in particular Aerosil grades, talc, modified starches and starch derivatives, crystalline cellulose, cellulose powders, lactoglobulin derivatives, microsponges, polymer particles made of nylon, polyolefins, polycarbonates, polyurethanes, polyacrylates, (meth)acrylate or (meth)acrylate-vinylidene copolymers that can be cross-linked, polyesters, polyamides, polystyrenes, Teflon, and silicones. A particularly preferred raw material of this kind is Vegetal Filling Spheres of Coletica.

Cosmetic emulsions particularly preferred according to the present invention are characterized in that they contain at least one cosmetic active substance c) that is selected from monomers, oligomers, and polymers of amino acids, N—C₂-C₂₄ acylamino acids, and/or the esters and/or physiologically compatible salts of said substances, in a total quantity from 0.000001 to 5 wt %, preferably 0.00001 to 2 wt %, particularly preferably 0.0001 to 1 wt %, and extraordinarily preferably 0.005 to 0.5 wt %, based in each case on the active substance content in the entire emulsion.

In a further preferred embodiment, the oil-in-water emulsions according to the present invention contain as a cosmetic active substance c) at least one DNA oligonucleotide or an RNA oligonucleotide. Positive effects are attributed to these components especially in the context of anti-wrinkle and anti-aging treatment.

According to the present invention, an “oligonucleotide” is understood as polymerizates of 2 to 20, preferably 2 to 10 mononucleotides that, as in the case of polynucleotides and nucleic acids, are linked by phosphoric acid diester bridges. The nucleotides are made up of nucleobases (usually derivatives of pyrimidine or purine), pentoses (usually D-ribofuranose or 2-deoxy-D-ribofuranose in a β-N-glycoside bond onto the nucleobase), and phosphoric acid. The mononucleotides are, for example, adenosine phosphates, cytidine phosphates, guanosine phosphates, uridine phosphates, and thymidine phosphates, in particular CMP (cytidine 5′-monophosphate), UDP (uridine 5′-diphosphate), ATP (adenosine 5′-triphosphate), and GTP (guanosine 5′-triphosphate).

An oligonucleotide that is particularly preferred according to the present invention is the thymidine dinucleotide.

Preferred oil-in-water emulsions according to the present invention are characterized in that they contain at least one DNA oligonucleotide or RNA oligonucleotide in a total quantity from 0.0001 to 5 wt %, preferably 0.001 to 1.0 wt %, and particularly preferably 0.01 to 0.5 wt %, based on the entire emulsion.

In a further preferred embodiment, the oil-in-water emulsions according to the present invention contain as a cosmetic active substance c) at least one natural betaine compound. These compounds have positive effects especially in the context of skin-moistening treatment. Natural betaine compounds that are preferred according to the present invention are naturally occurring compounds having the atomic grouping R₃N⁺—CH₂—X—COO⁻ according to IUPAC Rule C-816.1. Betaine surfactants (synthetic) are not included among the betaine compounds used according to the present invention; nor are other zwitterionic compounds in which the positive charge is located on N or P and the negative charge formally on O, S, B, or C, but that do not correspond to IUPAC Rule C-816.1. Betaine compounds preferred according to the present invention are betaine (Me₃N⁺—CH₂—COO⁻) and carnitine (Me₃N⁺—CH₂—CHOH—CH₂—COO⁻), where Me=methyl in each case.

Preferred oil-in-water emulsions according to the present invention are characterized in that they contain at least one natural betaine compound in a total quantity from 0.05 to 5 wt %, preferably 0.1 to 3 wt %, particularly preferably 0.5 to 2 wt %, based in each case on the entire emulsion.

In a further preferred embodiment, the oil-in-water emulsions according to the present invention contain as a cosmetic active substance c) at least one vitamin, provitamin, or a compound designated as a vitamin precursor, from the vitamin groups A, B, C, E, H, and K and the esters of the aforementioned substances. Positive effects are attributed to these components especially in the context of anti-wrinkle and anti-aging treatment, but also in the context of skin-moistening, lightening, sebum-regulating, and skin-calming treatment.

The group of substances referred to as vitamin A includes retinol (vitamin A₁) as well as 3,4-didehydroretinol (vitamin A₂). β-Carotene is the provitamin of retinol. Vitamin A components that are suitable according to the present invention are, for example, vitamin A acid and its esters, vitamin A aldehyde, and vitamin A alcohol, as well as esters thereof such as retinyl palmitate and retinyl acetate. The emulsions according to the present invention contain the at least one vitamin A component preferably in a total quantity from 0.05 to 1 wt % based on the entire emulsions.

Members of the vitamin B group or vitamin B complex are, among others:
Vitamin B₁, trivial name thiamine, chemical designation 3-[(4′-amino-2′-methyl-5′-pyrimidinyl)methyl]-5-(2-hydroxyethyl)-4-methylthiazolium chloride. Thiamine hydrochloride is preferably used in quantities from 0.05 to 1 wt %, based on the entire composition.
Vitamin B₂, trivial name riboflavin, chemical designation 7,8-dimethyl-10-(1-D-ribityl)benzo[g]pteridine-2,4(3H,10H)-dione. Riboflavin or its derivatives is preferably used in a total quantity from 0.05 to 1 wt %, based on the entire composition.
Vitamin B₃. The compounds nicotinic acid and nicotinic acid amide (niacinamide) are listed under this designation. Nicotinic acid amide is preferred according to the present invention; it is contained in the compositions according to the present invention preferably in quantities from 0.05 to 1 wt % based on the entire composition.
Vitamin B₅ (pantothenic acid and panthenol). Panthenol is preferably used. Derivatives of panthenol usable according to the present invention are, in particular, the esters and ethers of panthenol as well as cationically derivatized panthenols. in a further preferred embodiment of the invention, derivatives of 2-furanone having the general structural formula (VIT-I) can also be used instead of and in addition to pantothenic acid or panthenol.

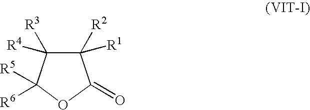

Those 2-furanone derivatives in which the substituents R¹ to R⁶, mutually independently, represent a hydrogen atom, a hydroxyl radical, a methyl, methoxy, aminomethyl, or hydroxymethyl radical, a saturated or mono- or diunsaturated, linear or branched C₂ to C₄ hydrocarbon radical, a saturated or mono- or diunsaturated, branched or linear mono-, di-, or trihydroxy-C₂ to C₄ hydrocarbon radical, or a mono- or diunsaturated, branched or linear mono-, di-, or triamino-C₂ to C₄ hydrocarbon radical, are preferred. Particularly preferred derivatives are the substances (also available commercially) dihydro-3-hydroxy-4,4-dimethyl-2(3H)-furanone having the trivial name pantolactone (Merck), 4-hydroxymethyl-γ-butyrolactone (Merck), 3,3-dimethyl-2-hydroxy-γ-butyrolactone (Aldrich), and 2,5-dihydro-5-methoxy-2-furanone (Merck), all stereoisomers expressly being included. The 2-furanone derivative that is extraordinarily preferred according to the present invention is pantolactone (dihydro-3-hydroxy-4,4-dimethyl-2(3H)-furanone), such that in the formula (VIT-I), R¹ denotes a hydroxyl group, R² a hydrogen atom, R³ and R⁴ a methyl group, and R⁵ and R⁶ a hydrogen atom. The (R)-pantolactone stereoisomer is produced upon breakdown of pantothenic acid.

The aforesaid compounds of the vitamin B₅ type, and the 2-furanone derivatives, are contained in the compositions according to the present invention preferably in a total quantity from 0.05 to 5 wt %, particularly preferably 0.1 to 3 wt %, extraordinarily preferably 0.5 to 2 wt %, based in each case on the entire composition.

Vitamin B₆, this being understood not as a uniform substance but as the derivatives, known under the trivial names pyridoxine, pyridoxamine, and pyridoxal, of 5-hydroxymethyl-2-methylpyridin-3-ol. Vitamin B₆ is contained in the compositions according to the present invention preferably in quantities from 0.0001 to 1.0 wt %, in particular in quantities from 0.001 to 0.01 wt %.

Vitamin B₇ (biotin), also referred to as vitamin H or “skin vitamin.” Biotin is (3aS,4S,6aR)-2-oxohexahydrothienol[3,4-d]-imidazole-4-valeric acid. Biotin is contained in the compositions according to the present invention preferably in quantities from 0.0001 to 1.0 wt %, in particular in quantities from 0.001 to 0.01 wt %.

Folic acid (vitamin B_{g}, vitamin B_{c}). International generic name for N-[4-(2-amino-3,4-dihydro-4-oxo-6-pteridinylmethylamino)benzoyl]-L-glutamic acid (N-pteroyl-L-glutamic acid, PteGlu). “Folate” is used synonymously with pteroyl glutamate; “folates” is the collective term for all folic-acid-active compounds, and designates a substance class that contains a pteridine ring joined to 4-aminobenzoic acid and L-glutamic acid. Folic acid is a growth factor for various microorganisms and a compound having vitamin characteristics, which occurs in nature usually as a polyglutamate and in reduced form (7,8-dihydrofolic acid, H₂-folate, DHF; tetrahydrofolic acid, H₄-folate, THF; 5′-methyltetrahydrofolic acid, CH₃—H₄-folate, MeTHF). Compositions particularly preferred according to the present invention are characterized in that they contain at least one component selected from folic acid, folates, and esters thereof, in a total quantity from 0.0001 to 1.0 wt %, in particular 0.01 to 0.5 wt %, based on the composition.

Orotic acid (vitamin B₁₃, 1,2,3,6-tetrahydro-2,6-dioxo-4-pyrimidinecarboxylic acid, uracil-6-carboxylic acid, whey acid). Orotic acid, its choline esters, or orotic acid metal salts (orotates of Ca, Cr, Fe, K, Co, Cu, Li, Mg, Mn, Na, Zn, Sn), are particularly preferred according to the present invention. Compositions particularly preferred according to the present invention are characterized in that they contain at least one component selected from orotic acid, orotates, and esters thereof, in a total quantity from 0.0001 to 1.0 wt %, in particular 0.01 to 0.5 wt %, based on the composition.

The vitamin C group includes vitamin C (ascorbic acid) and its derivatives, in particular the esters of ascorbic acid with organic and inorganic acids and salts thereof, as well as the acetals with glucose or other sugars, in particular ascorbyl glucoside. Vitamin C and/or at least one of its derivatives is used preferably in a total quantity from 0.1 to 3 wt %, based on the entire composition. Use of the derivatives ascorbyl palmitate, ascorbyl stearate, ascorbyl dipalmitate, ascorbyl acetate, Mg ascorbyl phosphate, Na ascorbyl phosphate, sodium and magnesium ascorbate, disodium ascorbyl phosphate and sulfate, potassium ascorbyltocopheryl phosphate, chitosan ascorbate, or ascorbyl glucoside may be preferred. The use of at least one member of the vitamin C group, in combination with tocopherols and/or other members of the vitamin E group, may likewise be preferred.

The vitamin E group includes tocopherol, in particular α-tocopherol, and its derivatives. Preferred derivatives are in particular the esters, such as tocopheryl acetate, tocopheryl nicotinate, tocopheryl phosphate, tocopheryl succinate, tocopheryl linoleate, tocopheryl oleate, tocophereth-5, tocophereth-10, tocophereth-12, tocophereth-18, tocophereth-50, and tocophersolan. Tocopherol and its derivatives are preferably contained in a total quantity from 0.05 to 1 wt %, based on the entire composition.

Vitamin His another term for biotin or vitamin B₇ (see above).

Among the fat-soluble vitamins of the vitamin K group, based on the fundamental structure of 2-methyl-1,4-naphthoquinone, are phylloquinone (vitamin K₁), famoquinone or menaquinone-7 (vitamin K₂), and menadione (vitamin K₃). Vitamin K is contained preferably in quantities from 0.0001 to 1.0 wt %, in particular 0.01 to 0.5 wt %, based in each case on the entire emulsion.

Vitamin A palmitate (retinyl palmitate), panthenol, pantolactone, nicotinic acid amide, pyridoxine, pyridoxamine, pyridoxal, biotin, ascorbyl palmitate and acetate, Mg ascorbyl phosphate, Na ascorbyl phosphate, sodium and magnesium ascorbate, and the tocopherol esters, especially tocopheryl acetate, are active substances c) that are particularly preferred according to the present invention.

In a further preferred embodiment, the oil-in-water emulsions according to the present invention contain as a cosmetic active substance c) at least one vitamin, provitamin, or a compound designated as a vitamin precursor from the vitamin groups A, B, C, E, H, and K and the esters of the aforesaid substances, in a total quantity from 0.1 to 5 wt %, by preference from 0.25 to 4 wt %, and in particular from 0.5 to 2.5 wt %, based in each case on the entire emulsion.

In a further preferred embodiment, the compositions according to the present invention contain at least one substance that is selected from the vitamins, provitamins, and vitamin precursors of the group B₁, B₂, B₃, B₆, B₇, B₉, B₁₃ and their esters and/or salts, and from pantolactone.

In a further preferred embodiment, the oil-in-water emulsions according to the present invention contain as a cosmetic active substance c) at least one α-hydroxycarboxylic acid, α-ketocarboxylic acid, or β-hydroxycarboxylic acid, or the ester, lactone, or salt form thereof. Positive effects are attributed to these components especially in the context of anti-wrinkle and anti-aging treatment, but also in the context of skin-moistening or moisture-donating, lightening, sebum-regulating, and anti-acne treatment. α-Hydroxycarboxylic acids or α-ketocarboxylic acid preferred according to the present invention are glycolic acid, lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2-hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, erythraric acid, threaric acid, glucaric acid, galactaric acid, mannaric acid, 2-hydroxy-2-methylsuccinic acid, gluconic acid, gularic acid, pyruvic acid, glucuronic acid, and galacturonic acid. Particularly preferred α-hydroxycarboxylic acids are lactic acid, citric acid, glycolic acid, and gluconic acid. A particularly preferred β-hydroxycarboxylic acid is salicylic acid. The esters of the aforesaid acids are preferably selected from the methyl, ethyl, propyl, isopropyl, butyl, amyl, pentyl, hexyl, 2-ethylhexyl, octyl, decyl, dodecyl, and hexadecyl esters. Particularly preferred emulsions according to the present invention are characterized in that at least one α-hydroxycarboxylic acid, α-ketocarboxylic acid, and/or β-hydroxycarboxylic acid and/or at least one derivative thereof is contained in a total quantity from 0.1 to 10 wt %, preferably 0.5 to 5 wt %, based in each case on the entire emulsion.

In a further preferred embodiment, the oil-in-water emulsions according to the present invention contain as a cosmetic active substance c) at least one flavonoid and/or at least one flavonoid-rich plant extract. Positive effects are attributed to these components especially in the context of anti-wrinkle and anti-aging treatment, but also in the context of skin-moistening or moisture-donating, lightening, sebum-regulating, and anti-acne treatment.

The flavonoids preferred according to the present invention encompass the glycosides of the flavones, of the flavanones, of 3-hydroxyflavone (flavonols), of the aurones, and of the isoflavones. Particularly preferred flavonoids are selected from naringin (aurantiin, naringenin-7-rhamnoglucoside), α-glucosylrutin, α-glucosylmyricetin, α-Glucosylisoquercetin, α-glucosylquercetin, hesperidin (3′,5,7-trihydroxy-4′-methoxyflavanone-7-rhamnoglucoside, hesperitin-7-O-rhamnoglucoside), neohesperidin, rutin (3,3′,4′,5,7-pentahydroxyflavone-3-rhamnoglucosid, quercetin-3-rhamnoglucoside), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), diosmin (3′,4′,7-trihydroxy-5-methoxyflavanone-7-rhamnoglucoside), eriodictin, and apigenin-7-glucoside (4′,5,7-trihydroxyflavone-7-glucoside).

Flavonoids that are extraordinarily preferred according to the present invention are α-glucosylrutin, naringin, and apigenin-7-glucoside.

Also preferred are the biflavonoids constructed from two flavonoid units, which occur e.g., in ginkgo species. Further preferred flavonoids are the chalcones, principally phloricin and neohesperidin dihydrochalcone.

Particularly preferred emulsions according to the present invention are characterized in that at least one flavonoid and/or at least one flavonoid-rich plant extract is contained in a total quantity from 0.0001 to 1 wt %, preferably 0.0005 to 0.5 wt %, and particularly preferably 0.001 to 0.1 wt %, based in each case on the flavonoid active substance in the entire emulsion.

In a further preferred embodiment, the oil-in-water emulsions according to the present invention contain as a cosmetic active substance c) at least one isoflavonoid or at least one isoflavonoid-rich plant extract. Included among the isoflavonoids at this juncture are the isoflavones and the isoflavone glycosides. Positive effects are attributed to these components especially in the context of anti-wrinkle and anti-aging treatment.

“Isoflavones” are to be understood for purposes of the present invention as substances that represent the hydrogenation, oxidation, or substitution products of 3-phenyl-4H-1-benzopyran; a hydrogenation can be present at the 2,3-position of the carbon structure, and oxidation can be present to form a carbonyl group in the 4-position; “substitution” is to be understood as the replacement of one or more hydrogen atoms by hydroxy or methoxy groups. Among the isoflavones preferred according to the present invention are, for example, daidzein, genistein, prunetin, biochanin, orobol, santal, pratensein, irigenin, glycitein, biochanin A and formononetin. Daidzein, genistein, glycitein, and formononetin are particularly preferred as isoflavones.

In the isoflavone glycosides preferred according to the present invention, the isoflavones are glycosidically linked via at least one hydroxy group to at least one sugar. Suitable sugars are mono- or oligosaccharides, in particular D-glucose, D-galactose, D-glucuronic acid, D-galacturonic acid, D-xylose, D-apiose, L-rhamnose, L-arabinose and rutinoise. Daidzin and genistin are particularly preferred isoflavone glycosides according to the present invention.

It is further preferred according to the present invention if the isoflavones and/or glycosides thereof are contained in the preparations as constituents of a substance mixture obtained from a plant, in particular of a plant extract. Plant-based substance mixtures of this kind can be obtained, in the manner commonly known to one skilled in the art, for example by being extracted or pressed out from plants such as soy, in particular from soybeans, red clover, or chickpeas. Particularly preferably, isoflavones or isoflavone glycosides are used in the preparations according to the present invention in the form of extracts obtained from soy, such as those commercially obtainable, for example, under the product designation Soy Protein Isolate SPI (Protein Technology International, St. Louis) or Soy Phytochemicals Concentrate SPC (Archer Daniels Midland, Decatur) A further particularly preferred isoflavonoid-rich plant extract is apple-core extract, in particular the commercial product Ederline of Seporga. Ederline contains phytohormones, isoflavonoids, phytosterols, triterpenoids, tocopherol, and natural waxes.

Particularly preferred emulsions according to the present invention are characterized in that at least one isoflavonoid and/or at least one isoflavonoid-rich plant extract is contained as a cosmetic active substance c), in a total quantity from 0.00001 to 1 wt %, preferably 0.0005 to 0.5 wt %, and particularly preferably 0.001 to 0.1 wt %, based in each case on the isoflavonoid active substance in the entire composition.

In a further preferred embodiment, the oil-in-water emulsions according to the present invention contain as a cosmetic active substance c) at least one polyphenol and/or at least one polyphenol-rich plant extract. Positive effects are attributed to these components especially in the context of anti-wrinkle, anti-aging, and sebum-regulating skin treatment.

“Polyphenols” are to be understood according to the present invention as aromatic compounds that contain at least two phenolic hydroxy groups in the molecule. These include the three dihydroxybenzenes catechol, resorcinol, and hydroquinone, furthermore phloroglucin, pyrogallol, and hexahydrobenzene. In nature, free and etherified polyphenols occur, for example, in blossom dyes (anthocyanidines, flavones), in tanning agents (catechins, tannins), as lichen or fern ingredients (usninic acid, acylpolyphenols), in lignins, and as gallic acid derivatives. Preferred polyphenols are flavones, catechins, usninic acid and, as tannins, the derivatives of gallic acid, digallic acid, and digalloylgallic acid. Particularly preferred polyphenols are the monomeric catechins, i.e., the derivatives of the flavan-3-ols, and leukoanthocyanidines, i.e., the derivatives of the leucoanthocyanidines that carry phenolic hydroxy groups preferably in the 5,7,3′,4′,5′-position, preferably epicatechin and epigallocatechin, as well as the tanning agents resulting therefrom by autocondensation. Tanning agents of this kind are preferably used not as isolated pure substance but as extracts of plant parts that are rich in tanning agents, e.g., extracts of catechu, quebracho, oak bark, and pine bark as well as other tree barks, leaves of green tea (Camellia sinensis), and maté. The tannins are likewise particularly preferred.

A particularly preferred polyphenol-rich cosmetic active substance is the commercial product Sepivinol R, an extract from red wine, obtainable from the Seppic company. A further particularly preferred polyphenol-rich cosmetic active substance is the commercial product Crodarom Chardonnay, an extract from the seeds of the Chardonnay grape, obtainable from the Croda company.

Particularly preferred emulsions according to the present invention are characterized in that at least one polyphenol and/or at least one polyphenol-rich plant extract is contained in a total quantity from 0.001 to 10 wt %, preferably 0.005 to 5 wt %, and particularly preferably 0.01 to 3 wt %, based in each case on the polyphenol active substance in the entire emulsion.

In a further preferred embodiment, the oil-in-water emulsions according to the present invention contain as a cosmetic active substance c) at least one ubiquinone and/or at least one ubiquinol and/or at least one derivative of said substances. Positive effects are attributed to these components especially in the context of anti-wrinkle and anti-aging treatment. Ubiquinols are the reduced form of the ubiquinones. The ubiquinones preferred according to the present invention have the formula (UBI-I):

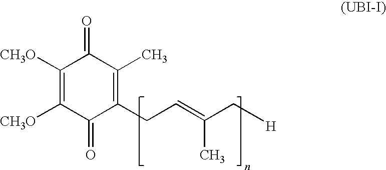

where n=6, 7, 8, 9, or 10.

The ubiquinone of formula (UBI-I) in which n=10, also known as Coenzyme Q10, is particularly preferred.

Particularly preferred emulsions according to the present invention are characterized in that at least one ubiquinone and/or at least one ubiquinol and/or at least one derivative of said substances is contained in a total quantity from 0.0001 to 1 wt %, preferably 0.001 to 0.5 wt %, and particularly preferably 0.005 to 0.1 wt %, based in each case on the entire emulsion.

In a further preferred embodiment, the oil-in-water emulsions according to the present invention contain silymarin as a cosmetic active substance c). Silymarin represents, according to the present invention, an active substance concentrate, previously considered a uniform substance, from the fruits of the milk thistle (Silybum marianum). The principal constituents of silymarin are silybin (silymarin I), silychristin (silymarin II), and silydianin, which belong to the group of the flavonolignans. Positive effects are attributed to these components especially in the context of skin-calming treatment.

Particularly preferred emulsions according to the present invention are characterized in that silymarin is contained in quantities from 0.0001 to 1 wt %, preferably 0.001 to 0.5 wt %, and particularly preferably 0.005 to 0.1 wt %, based in each case on the entire emulsion.

In a further preferred embodiment, the oil-in-water emulsions according to the present invention contain ectoin as a cosmetic active substance c). Positive effects are attributed to this component especially in the context of skin-moistening or moisture-donating treatment. Ectoin is the trivial name for 2-methyl-1,4,5,6-tetrahydropyrimidine-4-carboxylate. Particularly preferred emulsions according to the present invention are characterized in that ectoin is contained in quantities from 0.0001 to 1 wt %, preferably 0.001 to 0.5 wt %, and particularly preferably 0.005 to 0.01 wt %, based in each case on the entire composition.

In a further preferred embodiment, the oil-in-water emulsions according to the present invention contain as a cosmetic active substance c) at least one repellent, i.e., an active substance to repel insects.

Of the approximately fifteen active substances often used today in insect repellent agents, N,N-diethyl-3-methylbenzamide (DEET) is designated as the best all-around repellent. It has a repelling effect against mosquitoes, horse flies, sand flies, ticks, biting flies, mites, fleas, and bedbugs, the duration of action (as with all repellent active substances) being of different length with respect to the various species. Commercially available DEET preparations, for example, are effective for approx. 6 to 8 hours against mosquitoes, but only approx. 2 to 4 hours against ticks. A further common repellent active substance is 3-(N-n-butyl-N-acetylamino)propionic acid ethyl ester (also referred to as Repellent 3535). Repellent 3535 is effective against mosquitoes (Aedes aegypti, Anopheles albimanus), tsetse flies (Glossinae), and horse flies (Tabanidae). Also common is dimethyl phthalate (Palatinol M, DMP), which is effective against mosquitoes (especially Aedes and Anopheles species), lice, ticks, and mites, but is used predominantly in combination with additional repellent active substances.

In a further preferred embodiment, the oil-in-water emulsions according to the present invention contain as a cosmetic active substance c) at least one inorganic and/or at least one organic UV filter substance.

The UV filter substances are substances, present in liquid or crystalline fashion at room temperature, that are capable of absorbing ultraviolet radiation and re-emitting the absorbed energy in the form of longer-wave radiation, e.g., heat. A distinction is made between UVA filters and UVB filters. The UVA and UVB filters can be used both individually and in mixtures. The use of filter mixtures is preferred according to the present invention. The organic UV filters preferred according to the present invention are selected from the derivatives of dibenzoylmethane, cinnamic acid esters, diphenylacrylic acid esters, benzophenone, camphor, p-aminobenzoic acid esters, o-aminobenzoic acid esters, salicylic acid esters, benzimidazoles, symmetrically or asymmetrically substituted 1,3,5-triazines, monomeric and oligomeric 4,4-diarylbutadiene carboxylic acid esters and carboxylic acid amides, ketotricyclo(5.2.1.0)decane, benzalmalonic acid esters, benzoxazole, and any mixtures of the aforesaid components. The organic UV filters can be oil-soluble or water-soluble. The benzoxazole derivatives are advantageously present in dissolved form in the cosmetic preparations according to the present invention. It may be particularly preferred, if applicable, if the benzoxazole derivatives are present in pigmentary, i.e., undissolved form, for example at particle sizes from 10 nm to 300 nm. Oil-soluble UV filters that are particularly preferred according to the present invention are 1-(4-tert.-butylphenyl)-3-(4′-methoxyphenyl)propane-1,3-dione (Parsol® 1789), 1-phenyl-3-(4′-isopropylphenyl)propane-1,3-dione, 3-(4′-methylbenzylidene)-D,L-camphor, 4-(dimethylamino)benzoic acid 2-ethylhexyl ester, 4-(dimethylamino)benzoic acid 2-octyl ester, 4-(dimethylamino)benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (octocrylene), salicylic acid 2-ethylhexyl ester, salicylic acid 4-isopropylbenzyl ester, salicylic acid homomethyl ester (3,3,5-trimethylcyclohexyl salicylate), 2-hydroxy-4-methoxybenzophenone, 2-hydroxy-4-methoxy-4′-methylbenzophenone, 2,2′-dihydroxy-4-methoxybenzophenone, 2-(4′-diethylamino-2′-hydroxybenzoyl)benzoic acid hexyl ester (also: aminobenzophenone, obtainable under the designation Uvinul A Plus from the BASF company), 4-methoxybenzalmalonic acid di-2-ethylhexyl ester, UV filters bound to polymers, e.g., the 3-(4-(2,2-bis-ethoxycarbonylvinyl)phenoxy)propenyl)methoxysiloxane/dimethylsiloxane copolymer having the INCI name Dimethicodiethylbenzal Malonate (CAS no. 207574-74-1, Parsol® SLX), triazine derivatives such as, for example, 2,4-bis-{[4-(2-ethyl hexyloxy)-2-hydroxy]phenyl}-6-(4-methoxyphenyl)-1,3,5-triazine (INCI: Bis-Ethylhexyloxyphenol Methoxyphenyl Triazine, obtainable under the name Tinosorb S from CIBA), dioctylbutylamidotriazone (INCI: Diethylhexyl Butamido Triazone, obtainable under the name Uvasorb® HEB from Sigma 3V), 2,4,6-trianilino-(p-carbo-2′-ethyl-1′-hexyloxy)-1,3,5-triazine (Ethylhexyl Triazone, Uvinul® T 150), 2-[4,6-bis(2,4-dimethylphenyl)-1,3,5-triazin-2-yl]-5-(octyloxy)phenol (CAS no.: 2725-22-6), 2,4-bis-[5-1 (dimethylpropyl)benzoxazol-2-yl-(4-phenyl)imino]-6-(2-ethylhexyl)imino-1,3,5-triazine (CAS no. 288254-16-0, Uvasorb® K2A of 3V Sigma), the benzotriazole derivatives 2,2′-methylene-bis-(6-(2H-benzotriazol-2-yl)-4-(1,1,3,3-tetramethyl butyl)phenol) [Tinosorb M (Ciba)], 2,2′-methyl-bis-[6(2H-benzotriazol-2-yl)-4-(methyl)phenol] (MIXXIM BB/200 of the Fairmount Chemical company), 2-(2′-hydroxy-3′,5′-di-t-amylphenyl)benzotriazole (CAS no.: 025973-551), 2-(2′-hydroxy-5′-octylphenyl)benzotriazole (CAS no. 003147-75-9), 2-(2′-hydroxy-5′-methylphenyl)benzotriazole (CAS no. 2440-22-4), 2-(2H-benzotriazol-2-yl)-4-methyl-6-[2-methyl-3-[1,3,3,3-tetramethyl-1-((trimethylsilyl)oxy]disiloxanyl)propyl]phenol (CAS no.: 155633-54-8) having the INCI name: Drometrizole Trisiloxane, 2,4-bis-{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine or also Aniso Triazine, obtainable as Tinosorb® S from CIBA), 2,4-bis-{[4-(3-sulfonato)-2-hydroxypropyloxy)-2-hydroxy]phenyl}-6-(4-methoxyphenyl)-1,3,5-triazine sodium salt, 2,4-bis-{[4-(3-(2-propyloxy)-2-hydroxypropyloxy)-2-hydroxy]phenyl}-6-(4-methoxyphenyl)-1,3,5-triazine, 2,4-bis-{[4-(2-ethyl hexyloxy)-2-hydroxy]phenyl}-6-[4-(2-methoxyethylcarboxyl)phenylamino]-1,3,5-triazine, 2,4-bis-{[4-(3-(2-propyloxy)-2-hydroxypropyloxy)-2-hydroxy]phenyl}-6-[4-(ethylcarboxyl)phenylamino]-1,3,5-triazine, 2,4-bis-{[4-(2-ethylhexyloxy)-2-hydroxy]phenyl}-6-(1-methylpyrrol-2-yl)-1,3,5-triazine, 2,4-bis-{[4-tris(trimethylsiloxysilylpropyloxy)-2-hydroxy]phenyl}-6-(4-methoxyphenyl)-1,3,5-triazine, 2,4-bis-{[4-(2-methylpropenyloxy)-2-hydroxy]phenyl}-6-(4-methoxyphenyl)-1,3,5-triazine, 2,4-bis-{[4-(1′,1′,1′,3′,5′,5′,5′-heptamethylsiloxy-2-methylpropyloxy)-2-hydroxy]phenyl}-6-(4-methoxyphenyl)-1,3,5-triazine, and mixtures of the aforesaid components.

Preferred water-soluble UV filters are 2-phenylbenzimidazole-5-sulfonic acid, phenylene-1,4-bis-(2-benzimidazyl)-3,3′-5,5′-tetrasulfonic acid, and their alkali, alkaline-earth, ammonium, alkylammonium, alkanolammonium, and glucammonium salts, in particular the sulfonic acid itself having the INCI name Phenylbenzimidazole Sulfonic Acid (CAS no. 27503-81-7), which is obtainable e.g., under the commercial name Eusolex 232 from Merck or as Neo Heliopan Hydro from Symrise, and the phenylene-1,4-bis-(2-benzimidazyl)-3,3′-5,5′-tetrasulfonic acid bis-sodium salt having the INCI name Disodium Phenyl Dibenzimidazole Tetrasulfonate (CAS no.: 180898-37-7), which is obtainable e.g., under the commercial name Neo Heliopan AP from Symrise, sulfonic acid derivatives of benzophenones, by preference 2-hydroxy-4-methoxybenzophenone-5-sulfonic acid and its salts, sulfonic acid derivatives of 3-benzylidene camphor such as, for example, 4-(2-oxo-3-bornylidenemethyl)benzenesulfonic acid and 2-methyl-5-(2-oxo-3-bornylidene)sulfonic acid and salts thereof, having the INCI name Terephthalydene Dicamphor Sulfonic Acid (CAS no.: 90457-82-2, obtainable as Mexoryl SX from the Chimex company).

Some of the oil-soluble UV filters can themselves serve as solvents or solubilizers for other UV filters. For example, solutions of the UV-A filter 1-(4-tert.-butylphenyl)-3-(4′methoxyphenyl)propane-1,3-dione (e.g., Parsol® 1789) in various UV-B filters can be produced. The compositions according to the present invention, therefore, contain, in a further preferred embodiment, 1-(4-tert.-butylphenyl)-3-(4′-methoxyphenyl)propane-1,3-dione in combination with at least one UV-B filter selected from 4-methoxycinnamic acid 2-ethylhexyl ester, 2-cyano-3,3-phenylcinnamic acid 2-ethylhexyl ester, salicylic acid 2-ethylhexyl ester, and 3,3,5-trimethylcyclohexyl salicylate. In these combinations, the weight ratio of UV-B filter to 1-(4-tert-butylphenyl)-3-(4′-methoxyphenyl)propane-1,3-dione is between 1:1 and 10:1, preferably between 2:1 and 8:1; the molar ratio is correspondingly between 0.3 and 3.8, preferably between 0.7 and 3.0.

The inorganic light protection pigments preferred according to the present invention are finely dispersed or colloidally dispersed metal oxides and metal salts, for example titanium dioxide, zinc oxide, iron oxide, aluminum oxide, cerium oxide, zirconium oxide, silicates (talc), and barium sulfate. The particles should have an average diameter of less than 100 nm, by preference between 5 and 50 nm, and in particular between 15 and 30 nm (nanopigments). They can have a spherical shape, but it is also possible to use particles that possess an ellipsoidal shape or one that otherwise deviates from a spherical configuration. The pigments can also be surface-treated, i.e., hydrophilized or hydrophobized. Typical examples are coated titanium dioxides such as, for example, Titanium Dioxide T 805 (Degussa) or Eusolex® T2000 (Merck). Suitable hydrophobic coating agents are principally silicones and, in that context, especially trialkoxyoctylsilanes or simethicones. Titanium dioxide and zinc oxide are particularly preferred.

Preferred emulsions according to the present invention are characterized in that they contain at least one organic UV filter substance in a total quantity from 0.1 to 30 wt %, preferably 0.5 to 20 wt %, particularly preferably 1.0 to 10 wt %, and extraordinarily preferably 2 or 3 to 7 wt %, based in each case on the entire composition.

Preferred compositions according to the present invention are characterized in that they contain at least one inorganic UV filter substance in a total quantity from 0.1 to 15 wt %, preferably 0.5 to 10 wt %, particularly preferably 1 to 5 wt %, and extraordinarily preferably 2 to 4 wt %, based in each case on the entire composition.

In a further preferred embodiment, the oil-in-water emulsions according to the present invention contain as a cosmetic active substance c) at least one self-tanning active substance. Self-tanning active substances preferred according to the present invention are selected from dihydroxyacetone, erythrulose, and 5,6-dihydroxyindoline as well as mixtures of said components, in particular mixtures of dihydroxyacetone and erythrulose.

Preferred emulsions according to the present invention are characterized in that they contain at least one self-tanning active substance in a total quantity from 0.01 to 15 wt %, preferably 0.1 to 10 wt %, particularly preferably 1.0 to 5 wt %, and extraordinarily preferably 2.0 to 4.0 wt %, based in each case on the entire emulsion.

In a further preferred embodiment, the oil-in-water emulsions according to the present invention contain as a cosmetic active substance c) at least one skin-lightening active substance. Skin-lightening active substances preferred according to the present invention are selected from ascorbic acid, the esters of ascorbic acid with phosphoric acid and/or organic C₂ to C₂₀ carboxylic acids, as well as their alkali and alkaline-earth metal salts, kojic acid, hydroquinone, arbutine, mulberry tree extract, and licorice extract, as well as mixtures thereof. The ascorbic acid derivatives and kojic acid are preferred, both as individual substances and mixed. Sodium ascorbyl phosphate, magnesium ascorbyl phosphate, ascorbyl monopalmitate, ascorbyl dipalmitate, ascorbyl monostearate, ascorbyl distearate, ascorbyl monoethylhexanoate, ascorbyl diethylhexanoate, ascorbyl monooctanoate, ascorbyl dioctanoate, ascorbyl monoisostearate, and ascorbyl diisostearate are particularly preferred. The ascorbic acid derivatives that are extraordinarily preferred according to the present invention are sodium ascorbyl phosphate and magnesium ascorbyl phosphate.

Preferred emulsions according to the present invention are characterized in that they contain at least one skin-lightening active substance in a total quantity from 0.05 to 5 wt %, preferably 0.1 to 2 wt %, based in each case on the entire emulsion.

In a further preferred embodiment, the oil-in-water emulsions according to the present invention contain as a cosmetic active substance c) at least one skin-calming active substance. Skin-calming substances preferred according to the present invention are selected from allantoin, α-bisabolol, α-liponic acid, extracts of Centella asiatica, obtainable e.g., under the designation Madecassicoside from DSM, glycyrrhetinic acid, which particularly preferably is present encapsulated in liposomes and in this form is obtainable e.g., under the commercial name Calmsphere from Soliance, mixtures of grain waxes, extracts of shea butter, and Argania spinosa oil having the INCI name “Spent grain wax and Butyrospermum Parkii (shea butter) extract and Argania Spinosa Kernel Oil,” as available e.g., under the commercial designation Stimu-Tex AS from the Pentapharm company, extracts of Vanilla tahitensis such as those obtainable e.g., under the commercial designation Vanirea (INCI: Vanilla Tahitensis Fruit Extract) from the Solabia company, algin hydrolysates such as those obtainable e.g., under the commercial designation Phycosaccharide, in particular Phycosaccharide Al, from the Codif company, extracts of Bacopa monniera such as those obtainable e.g., under the commercial designation Bacocalmine from the Sederma company, extracts from the rooibos plant such as those obtainable e.g., under the commercial name Rooibos Herbasec MPE from the Cosmetochem company, yeast extracts, particularly preferably the commercial product Drieline (INCI name: “Sorbitol, Yeast Extract”), obtainable from the Lanatech company, the physiologically compatible salts of sterol sulfates such as those obtainable e.g., under the commercial designation Phytocohesine (INCI: Sodium Beta-Sitosterylsulfate) from the Vincience company, aminodicarboxylic acids having a carbon chain length from 3 to 6 carbon atoms and their physiologically compatible salts, preferably selected from aminomalonic acid, aminosuccinic acid (=aspartic acid), aminoglutaric acid, and aminoadipic acid as well as their physiologically compatible salts such as potassium aspartate and magnesium aspartate, as well as any mixtures of said substances.

Further preferred emulsions according to the present invention are characterized in that they contain at least one skin-calming active substance in a total quantity from 0.001 to 5 wt %, preferably 0.01 to 2 wt %, and particularly preferably 0.1 to 1 wt %, based in each case on the entire emulsion.

In a further embodiment, the oil-in-water emulsions according to the present invention contain as a cosmetic active substance c) at least one moisture-donating active substance. Moisture-donating active substances preferred according to the present invention are selected from deoxy sugars, particularly preferably rhamnose and fucose, polysaccharides that contain at least one deoxy sugar module, particularly preferably from the commercial products Fucogel® (INCI name: Biosaccharide Gum-1) of Solabia, Rhamnosoft® (INCI name: Biosaccharide Gum-2) of Solabia, Fucogenol® (INCI name: Biosaccharide Gum-3) of Solabia, and Glycofilm® (INCI name: Biosaccharide Gum-4) of Solabia, also mixtures of the aforesaid polysaccharides containing at least one deoxy sugar module, for example the mixture of Biosaccharide Gum-2 and Biosaccharide Gum-3 obtainable as a commercial product Elastinol Plus® from Solabia, furthermore urea, N,N′-bis(2-hydroxyethyl)urea (obtainable, for example, under the commercial name Hydrovance), betaine (Me₃N⁺—CH₂—COO⁻), glycosaminoglycans, particularly preferably hyaluronic acid, dextran, dextran sulfate, chondroitin 4-sulfate and chondroitin 6-sulfate, as well as any mixtures of said substances.

Preferred emulsions according to the present invention are characterized in that they contain at least one moisture-donating active substance in a total quantity from 0.001 to 10 wt %, preferably 0.1 to 5 wt %, and particularly preferably 1 to 3 wt %, based in each case on the entire emulsion.

In a further preferred embodiment, the oil-in-water emulsions according to the present invention contain as a cosmetic active substance c) at least one sebum-regulating active substance. Sebum-regulating active substances preferred according to the present invention are selected from azelaic acid, azelaic acid derivatives, in particular the azelaic acid derivative potassium azeloyl diglycinate, which is obtainable e.g., as a commercial product Azeloglicina from Sinerga, sebacic acid, 10-hydroxydecanoic acid, 1,10-decanediol, mixtures of sebacic acid, 10-hydroxydecanoic acid, and 1,10-decanediol such as those obtainable, for example, as a commercial product Acnacidol PG from Vincience, glycyrrhizin, which is also referred to as glycyrrhizic acid or glycyrrhetinic acid glycoside and represents the 2-beta-glucuronido-alpha-glucuronide of glycyrrhetinic acid, as well as salts thereof, tannic acid and salts thereof, gallotannins, naringin, mixtures of glycyrrhizin (salts), tannic acid (salts), and/or gallotannins and naringin, such as those obtainable e.g., as a commercial product BiSCos Glynarin PF from the Biesterfeld company, additionally from extracts of Spiraea ulmaria such as those contained, for example, in the product Seboregul of the Silab company, additionally from water- and oil-soluble extracts of hamamelis, burdock root, and nettle, cinnamon tree extract (e.g., Sepicontrol® A5 of the Seppic company), chrysanthemum extract (e.g., Laricyl® of Laboratoires Serobiologiques), wheat protein hydrolysates such as those obtainable e.g., in the commercial products of the Asebiol® series of Laboratoires Serobiologiques, in particular Asebiol® LS 2539 BT 2 (INCI: Aqua, Hydrolyzed Yeast Protein, Pyridoxine, Niacinamide, Glycerin, Panthenol, Allantoin, Biotin) and Asebiol® LS 2539 BT (Aqua, Hydrolyzed Yeast Protein, Pyridoxine, Niacinamide, Glycerin, Panthenol, Propylene Glycol, Allantoin, Disodium Azelate, Biotin) and PEG-8 isolauryl thioether as contained e.g., in the commercial product Sebum Control® COS-218/2-A of Cosmetochem (INCI: Aqua, Cetyl-PCA, PEG-8 Isolauryl Thioether, PCA, Cetyl Alcohol).

Preferred emulsions according to the present invention are characterized in that they contain at least one sebum-regulating active substance in a total quantity from 0.0001 to 5 wt %, preferably 0.001 to 2 wt %, particularly preferably 0.01 to 1 wt %, and extraordinarily preferably 0.1 to 0.5 wt %, based in each case on the active substance content in the entire emulsion according to the present invention.

Further oil-in-water emulsions preferred according to the present invention are characterized in that the oil phase or fat phase encompasses at least one fragrance.

Perfumes, perfume oils, or perfume oil constituents can be used as fragrance components. Perfume oils or fragrances can be, according to the present invention, individual odorant compounds, e.g., synthetic products of the ester, ether, aldehyde, ketone, alcohol, and hydrocarbon types. Odorant compounds of the ester type are, for example, benzyl acetate, phenoxyethyl isobutyrate, p-tert.-butyl cyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, floramate, melusate, and jasmecyclate. The ethers include, for example, benzyl ethyl ether and ambroxan; the aldehydes, for example, the linear alkanals having 8 to 18 carbon atoms, citral, citronellal, citronellyl oxyacetaldehyde, cyclamenaldehyde, lilial and bourgeonal; the ketones, for example, the ionones, α-isomethylionone and methyl cedryl ketone; the alcohols, anethol, citronellol, eugenol, geraniol, linalool, phenylethyl alcohol and terpineol; and the hydrocarbons include principally the terpenes such as limonene and pinene. Preferably, however, mixtures of different odorants that together produce an attractive fragrance note are used.

Such perfume oils can also contain natural odorant mixtures such as those accessible from plant sources, for example pine, citrus, jasmine, patchouli, rose, or ylang-ylang oil. Also suitable are muscatel sage oil, chamomile oil, clove oil, lemon balm oil, mint oil, cinnamon leaf oil, linden blossom oil, juniper berry oil, vetiver oil, olibanum oil, galbanum oil, and labdanum oil, as well as orange blossom oil, neroli oil, orange peel oil, and sandalwood oil.

In order to be perceptible, an odorant must be volatile; in addition to the nature of the functional groups and the structure of the chemical compound, the molecular weight also plays an important part. Most odorants, for example, possess molar weights of up to approximately 200 dalton, while molar weights of 300 dalton and above represent something of an exception. Because of the differing volatility of odorants, the odor of a perfume or fragrance made up of multiple odorants changes during volatilization, the odor impressions being subdivided into a “top note,” “middle note” or “body,” and “end note” or “dry out.” Because the perception of an odor also depends a great deal on the odor intensity, the top note of a perfume or fragrance is not made up only of highly volatile compounds, while the end note comprises for the most part less-volatile, i.e., adherent odorants. In the compounding of perfumes, more-volatile odorants can, for example, be bound to specific fixatives, thereby preventing them from volatilizing too quickly. Therefore, in the division below of odorants into “more-volatile” and “adherent” odorants, no statement is made with regard to the odor impression, or as to whether the corresponding odorant is perceived as a top or middle note.

Adherent odorants that are usable in the context of the present invention are, for example, the essential oils such as angelica oil, anise oil, arnica flower oil, basil oil, bay oil, bergamot oil, champaca flower oil, silver fir oil, silver fir cone oil, elemi oil, eucalyptus oil, fennel oil, fir needle oil, galbanum oil, geranium oil, gingergrass oil, guaiac wood oil, balsam gurjun oil, helichrysum oil, ho oil, ginger oil, iris oil, cajeput oil, calamus oil, chamomile oil, camphor oil, kanaga oil, cardamom oil, cassia oil, pine needle oil, balsam copaiva oil, coriander oil, curled peppermint oil, caraway oil, cumin oil, lavender oil, lemon grass oil, lime oil, tangerine oil, lemon balm oil, ambrette seed oil, myrrh oil, clove oil, neroli oil, niaouli oil, olibanum oil, orange oil, oregano oil, palmarosa oil, patchouli oil, balsam peru oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, vetiver oil, juniper berry oil, wormwood oil, wintergreen oil, ylang-ylang oil, ysop oil, cinnamon oil, cinnamon leaf oil, citronella oil, lemon oil, and cypress oil.

The higher-boiling or solid odorants of natural or synthetic origin can, however, also be used in the context of the present invention as adherent odorants or odorant mixtures, i.e., fragrances. These compounds include the compounds recited below, as well as mixtures thereof: ambrettolide, α-amyl cinnamaldehyde, anethol, anisealdehyde, anise alcohol, anisol, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bornyl acetate, α-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvacrol, carvone, p-cresol methyl ether, cumarin, p-methoxyacetophenone, methyl n-amyl ketone, methylanthranilic acid methyl ester, p-methyl acetophenone, methylchavicol, p-methyl quinoline, methyl-β-naphthyl ketone, methyl n-nonylacetaldehyde, methyl n-nonyl ketone, muscone, β-naphthol ethyl ether, β-naphthol methyl ether, nerol, nitrobenzene, n-nonylaldehyde, nonyl alcohol, n-octylaldehyde, p-oxyacetophenone, pentadecanolide, β-phenylethyl alcohol, phenylacetaldehyde dimethyl acetal, phenylacetic acid, pulegone, safrol, salicylic acid isoamyl ester, salicylic acid methyl ester, salicylic acid hexyl ester, salicylic acid cyclohexyl ester, santalol, skatole, terpineol, thymene, thymol, γ-undelactone, vanillin, veratrumaldehyde, cinnamaldehyde, cinnamyl alcohol, cinnamic acid, cinnamic acid ethyl ester, cinnamic acid benzyl ester.

Included among the more-volatile odorants are, in particular, the lower-boiling odorants of natural or synthetic origin, which can be used alone or in mixtures. Examples of more-volatile odorants are alkyl isothiocyanates (alkylmustard oils), butadione, citral, citronellal, limonene, linalool, linalyl acetate and propionate, menthol, menthone, methyl-n-heptenone, phellandrene, phenylacetaldehyde, and terpinyl acetate.

Particularly preferred cosmetic emulsions according to the present invention are characterized in that at least one fragrance component is contained in a total quantity from 0.00001 to 4 wt %, preferably 0.5 to 2 wt %, particularly preferably 1 to 1.5 wt %, based in each case on the entire composition.

Further oil-in-water emulsions preferred according to the present invention are characterized in that they are packaged in a container having a ball applicator or roll-on applicator.

A further subject of the present invention is the cosmetic, non-therapeutic use of an oil-in-water emulsion in which the cosmetic active substance c) is selected from perspiration-inhibiting active substances, for perspiration-inhibiting treatment of the skin, in particular the armpit skin and/or foot skin.

A further subject of the present invention is the cosmetic, non-therapeutic use of an oil-in-water emulsion in which the cosmetic active substance c) is selected from perspiration-inhibiting active substances, for perspiration-inhibiting treatment of the skin, in particular the armpit skin and/or foot skin, with a non-greasy skin feel.

A further subject of the present invention is the cosmetic, non-therapeutic use of an oil-in-water emulsion in which the cosmetic active substance c) is selected from perspiration-inhibiting active substances, for perspiration-inhibiting treatment of the skin, in particular the armpit skin and/or foot skin, with accelerated drying.

A further subject of the present invention is a cosmetic, non-therapeutic method for perspiration-inhibiting treatment of the skin, in particular the armpit skin and/or foot skin, that is characterized in that an oil-in-water emulsion in which the cosmetic active substance c) is selected from perspiration-inhibiting active substances, is applied in an effective quantity onto the skin.

A further subject of the present invention is the cosmetic, non-therapeutic use of an oil-in-water emulsion in which the cosmetic active substance c) is selected from deodorizing active substances, monomers, oligomers, and polymers of amino acids, N—C₂-C₂₄ acylamino acids, the esters and/or the physiologically compatible salts of said substances, DNA or RNA oligonucleotides, natural betaine compounds, vitamins, provitamins, and vitamin precursors of groups A, B, C, E, H, and K, and the esters of the aforesaid substances, α-hydroxycarboxylic acids, α-ketocarboxylic acids, β-hydroxycarboxylic acids, and their ester, lactone, or salt form, flavonoids and flavonoid-rich plant extracts, isoflavonoids and isoflavonoid-rich plant extracts, polyphenols and polyphenol-rich plant extracts, ubiquinone and ubiquinol, and derivatives thereof, silymarin, ectoin, repellents, inorganic and organic UV-filtering substances, self-tanning active substances, skin-lightening active substances, skin-calming active substances, moisture-donating active substances, and sebum-regulating active substances, for deodorizing, anti-wrinkle, anti-aging, anti-pimple, anti-acne, sebum-regulating, skin-moistening or moisture-donating, light protecting, insect-repelling, self-tanning, or lightening treatment of the skin.

A further subject of the present invention is the cosmetic, non-therapeutic use of an oil-in-water emulsion in which the cosmetic active substance c) is selected from deodorizing active substances, monomers, oligomers, and polymers of amino acids, N—C₂-C₂₄ acylamino acids, the esters and/or the physiologically compatible salts of said substances, DNA or RNA oligonucleotides, natural betaine compounds, vitamins, provitamins, and vitamin precursors of groups A, B, C, E, H, and K, and the esters of the aforesaid substances, α-hydroxycarboxylic acids, α-ketocarboxylic acids, β-hydroxycarboxylic acids, and their ester, lactone, or salt form, flavonoids and flavonoid-rich plant extracts, isoflavonoids and isoflavonoid-rich plant extracts, polyphenols and polyphenol-rich plant extracts, ubiquinone and ubiquinol, and derivatives thereof, silymarin, ectoin, repellents, inorganic and organic UV-filtering substances, self-tanning active substances, skin-lightening active substances, skin-calming active substances, moisture-donating active substances, and sebum-regulating active substances, for deodorizing, anti-wrinkle, anti-aging, anti-pimple, anti-acne, sebum-regulating, skin-moistening or moisture-donating, light protecting, insect-repelling, self-tanning, or lightening treatment of the skin, with a non-greasy feel.

A further subject of the present invention is the cosmetic, non-therapeutic use of an oil-in-water emulsion in which the cosmetic active substance c) is selected from deodorizing active substances, monomers, oligomers, and polymers of amino acids, N—C₂-C₂₄ acylamino acids, the esters and/or the physiologically compatible salts of said substances, DNA or RNA oligonucleotides, natural betaine compounds, vitamins, provitamins, and vitamin precursors of groups A, B, C, E, H, and K, and the esters of the aforesaid substances, α-hydroxycarboxylic acids, α-ketocarboxylic acids, β-hydroxycarboxylic acids, and their ester, lactone, or salt form, flavonoids and flavonoid-rich plant extracts, isoflavonoids and isoflavonoid-rich plant extracts, polyphenols and polyphenol-rich plant extracts, ubiquinone and ubiquinol, and derivatives thereof, silymarin, ectoin, repellents, inorganic and organic UV-filtering substances, self-tanning active substances, skin-lightening active substances, skin-calming active substances, moisture-donating active substances, and sebum-regulating active substances, for deodorizing, anti-wrinkle, anti-aging, anti-pimple, anti-acne, sebum-regulating, skin-moistening or moisture-donating, light protecting, insect-repelling, self-tanning, or lightening treatment of the skin, with accelerated drying.

A further subject of the present invention is a cosmetic, non-therapeutic method for deodorizing, anti-wrinkle, anti-aging, anti-pimple, anti-acne, sebum-regulating, skin-moistening or moisture-donating, light protecting, insect-repelling, self-tanning, or lightening treatment of the skin, which method is characterized in that an oil-in-water emulsion in which the cosmetic active substance c) is selected from deodorizing active substances, monomers, oligomers, and polymers of amino acids, N—C₂-C₂₄ acylamino acids, the esters and/or the physiologically compatible salts of said substances, DNA or RNA oligonucleotides, natural betaine compounds, vitamins, provitamins, and vitamin precursors of groups A, B, C, E, H, and K, and the esters of the aforesaid substances, α-hydroxycarboxylic acids, α-ketocarboxylic acids, β-hydroxycarboxylic acids, and their ester, lactone, or salt form, flavonoids and flavonoid-rich plant extracts, isoflavonoids and isoflavonoid-rich plant extracts, polyphenols and polyphenol-rich plant extracts, ubiquinone and ubiquinol, and derivatives thereof, silymarin, ectoin, repellents, inorganic and organic UV-filtering substances, self-tanning active substances, skin-lightening active substances, skin-calming active substances, moisture-donating active substances, and sebum-regulating active substances, is applied in an effective quantity onto the skin.

### Manufacturing Emulsions According to the Present Invention.

The emulsions according to the present invention can be manufactured in accordance with a variety of manufacturing methods. Manufacturing methods preferred according to the present invention are presented below.

### Method 1.

Approximately a third of the total quantity of water (phase 1), and the oil component(s) i) to iii) and the emulsifier(s) that may optionally be contained, as well as any further oil or fat components (phase 2), are heated separately from one another to a temperature between 70 and 80° C. Phase 1 is then slowly added to phase 2, and the whole is emulsified at low rotation speed, and homogenized at low intensity, for 45 to 60 minutes. The batch is then cooled to 40 to 45° C. The cosmetic raw material c) (if sufficiently temperature-stable) and 15 to 20 wt % of the total quantity of water are then heated to 40 to 45° C., added to the batch, and homogenized. The remaining portion of the total quantity of water, together with the polysaccharide and any temperature-sensitive active substances c) and additives, for example preservatives, are then added to the batch, homogenized at high rotation speed, and cooled to 25° C. with slow stirring.

The perspiration-inhibiting active substances are not temperature-sensitive under the aforesaid manufacturing conditions, and can be incorporated at 40 to 50° C.

The duration of the individual homogenization steps is 0.5 to 10 minutes, preferably 1 to 8 minutes, particularly preferably 2 to 5 minutes.

### Method 2.

Approximately a third of the total quantity of water (phase 1), and the oil component(s) i) to iii) and the emulsifier(s) that may optionally be contained, as well as any further oil or fat components (phase 2), are heated separately from one another to a temperature between 70 and 80° C. Phase 2 is then slowly added to phase 1, and the whole is emulsified at low rotation speed and homogenized at low intensity, for 45 to 60 minutes. The batch is then cooled to 40 to 45° C. The cosmetic raw material c) (if sufficiently temperature-stable) and 15 to 20 wt % of the total quantity of water are then heated to 40 to 45° C., added to the batch, and homogenized. The remaining portion of the total quantity of water, together with the polysaccharide and any temperature-sensitive active substances c) and additives, for example preservatives, are then added to the batch, homogenized at high rotation speed, and cooled to 25° C. with slow stirring.

The perspiration-inhibiting active substances are not temperature-sensitive under the aforesaid manufacturing conditions, and can be incorporated at 40 to 50° C.

The duration of the individual homogenization steps is 0.5 to 10 minutes, preferably 1 to 8 minutes, particularly preferably 2 to 5 minutes.

### Method 3.

Approximately 20% of the total quantity of water, together with the oil component(s) i) to iii) and the emulsifier(s) that may optionally be contained, are heated to a temperature between 70 and 80° C., and emulsified at low rotation speed and homogenized at low intensity.

A further 10 to 20% of the total quantity of water is then heated to 70 to 80° C. and added; the whole is homogenized at high rotation speed and then emulsified for 0.5 to 2 hours. A further 10 to 20% of the total quantity of water is then heated to 70 to 80° C. and added, and the whole is homogenized at high rotation speed. The batch is cooled to 40 to 50° C. A portion of the cosmetic raw material c) is then heated to 40 to 50° C. (provided such raw material is stable at that temperature) along with a further 10 to 20% of the total quantity of water, and added to the batch, and the whole is homogenized at high rotation speed. The remaining portion of the cosmetic raw material c) is then heated to 40 to 50° C. (provided such raw material is stable at that temperature) along with a further 10 to 20% of the total quantity of water, and added to the batch, and the whole is homogenized at high rotation speed.

The batch is then cooled to 30 to 35° C. The remaining portion of the total quantity of water is then added, homogenized at high rotation speed, and slowly cooled while stirring.

If the cosmetic raw material c) is temperature-sensitive, it is added to the batch only together with the polysaccharide and, if applicable, further (temperature-sensitive) additives, for example preservatives, and the whole is homogenized at high rotation speed and cooled to 25° C. with slow stirring.

The perspiration-inhibiting active substances are not temperature-sensitive under the aforesaid manufacturing conditions, and can be incorporated at 40 to 50° C. for all the methods.

The duration of the individual homogenization steps for all the methods is 0.5 to 10 minutes, preferably 1 to 8 minutes, particularly preferably 2 to 5 minutes.

A low shear rate is by definition in the range from 1,000 to 2,500 revolutions of the stirring element per minute. A high shear rate is by definition in the range from 3,000 to 6,000 revolutions of the stirring element per minute.

The Examples below are intended to explain further the subject matter of the invention, but without limiting it thereto.

### EXAMPLE 1

### Antiperspirant Emulsion (O/W) According to the Present Invention with 1.8 wt % Oil and Fat Phase

| INGREDIENTS (EU/INCI) | Proportion (wt %) | |
|---|---|---|
| AQUA | 81.0 | |
| ALUMINUM CHLOROHYDRATE | 13.0 | |
| STEARETH-2 | 2.5 | |
| STEARETH-21 | 1.5 | |
| PERFUME | 1.1 | oil |
| PPG-15 STEARYL ETHER | 0.5 | oil |
| BISABOLOL | 0.1 | oil |
| ALUMINUM STARCH OCTENYLSUCCINATE | 0.1 | |
| BIS-PEG-18 METHYL ETHER DIMETHYL | 0.1 | fat |
| SILANE | | |
| TOCOPHERYL ACETATE | 0.1 | oil |

The emulsion according to Example 1 had, on the first day after manufacture, a viscosity of 1,800 mPas measured with a Brookfield viscosimeter, RV 4 spindle, 20 s⁻¹, without Helipath, at 20° C. ambient temperature and 20° C. sample temperature.

### EXAMPLE 2

### Antiperspirant Emulsion (O/W) According to the Present Invention with 1.5 wt % Oil and Fat Phase

| INGREDIENTS (EU/INCI) | Proportion (wt %) | |
|---|---|---|
| AQUA | 68.3 | |
| ALUMINUM CHLOROHYDRATE | 26.0 | |
| STEARETH-2 | 2.4 | |
| STEARETH-21 | 1.5 | |
| PERFUME | 1.0 | oil |
| PPG-15 STEARYL ETHER | 0.5 | oil |
| ALLANTOIN | 0.1 | |
| ALUMINUM STARCH OCTENYLSUCCINATE | 0.1 | |
| ALOE BARBADENSIS | 0.1 | |

The emulsion according to Example 2 had, on the first day after manufacture, a viscosity of 2,000 mPas measured with a Brookfield viscosimeter, RV 4 spindle, 20 s⁻¹, without Helipath, at 20° C. ambient temperature and 20° C. sample temperature.

### EXAMPLE 3

### Antiperspirant Emulsion (O/W) According to the Present Invention with 2.2 wt % Oil and Fat Phase

| INGREDIENTS (EU/INCI) | Proportion (wt %) | |
|---|---|---|
| AQUA | 90.0 | |
| ALUMINUM ZIRCONIUM | 23.7 | |
| TETRACHLOROHYDREX GLY | | |
| STEARETH-2 | 2.4 | |
| STEARETH-21 | 1.6 | |
| PERFUME | 1.2 | Oil |
| PPG-15 STEARYL ETHER | 0.5 | Oil |
| ALUMINUM STARCH OCTENYLSUCCINATE | 0.1 | |
| TOCOPHERYL ACETATE | 0.5 | Oil |

The emulsion according to Example 3 had, on the first day after manufacture, a viscosity of 2200 mPas measured with a Brookfield viscosimeter, RV 4 spindle, 20 s⁻¹, without Helipath, at 20° C. ambient temperature and 20° C. sample temperature.

### EXAMPLE 4

### Antiperspirant Emulsion (O/W) According to the Present Invention with 1.6 wt % Oil and Fat Phase

| INGREDIENTS (EU/INCI) | Proportion (wt %) | |
|---|---|---|
| AQUA | 74.4 | |
| ALUMINUM CHLOROHYDRATE | 20.0 | |
| STEARETH-2 | 2.3 | |
| STEARETH-21 | 1.5 | |
| PERFUME | 1.0 | oil |
| PPG-15 STEARYL ETHER | 0.5 | oil |
| ALUMINUM STARCH OCTENYLSUCCINATE | 0.1 | |
| ALLANTOIN | 0.1 | |
| ISOPROPYL MYRISTATE | 0.1 | oil |

The emulsion according to Example 4 had, on the first day after manufacture, a viscosity of 1700 mPas measured with a Brookfield viscosimeter, RV 4 spindle, 20 s⁻¹, without Helipath, at 20° C. ambient temperature and 20° C. sample temperature.

### EXAMPLE 5

### Antiperspirant Emulsion (O/W) According to the Present Invention with 1.6 wt % Oil and Fat Phase

| INGREDIENTS (EU/INCI) | Proportion (wt %) | |
|---|---|---|
| AQUA | 74.4 | |
| ALUMINUM CHLOROHYDRATE | 20.0 | |
| STEARETH-2 | 2.3 | |
| STEARETH-21 | 1.5 | |
| PERFUME | 1.0 | oil |
| PPG-15 STEARYL ETHER | 0.5 | oil |
| DISTARCH PHOSPHATE | 0.1 | |
| ALLANTOIN | 0.1 | |
| ISOPROPYL MYRISTATE | 0.1 | oil |

The emulsion according to Example 5 had, on the first day after manufacture, a viscosity of 1800 mPas measured with a Brookfield viscosimeter, RV 4 spindle, 20 s⁻¹, without Helipath, at 20° C. ambient temperature and 20° C. sample temperature.

The emulsions according to Examples 1 to 5 were each introduced into a bottle having a roll-on applicator and were thus ready for sale.

### Shelf Stability.

All the example emulsions according to the present invention were stable for 12 weeks when stored at 40° C.

All the example emulsions according to the present invention were stable for six weeks when stored at 45° C. When stored at 50° C., a slight creaming was evident, depending on the perfume oil, between the third and fourth week of storage. Without the critical perfume oils, however, these emulsions were shelf-stable at 50° C.

### Investigations of Drying Time.

The composition according to the present invention of Example 4 was compared with two comparison emulsions, not according to the present invention, having the following compositions:

### COMPARISON EXAMPLES C1 AND C2

### Antiperspirant Emulsions (O/W) not According to the Present Invention, without Polysaccharide (Quantities in wt %)

| INGREDIENTS (EU/INCI) | C1 | C2 |
|---|---|---|
| AQUA | 74.5 | 72.8 |
| ALUMINUM CHLOROHYDRATE | 20.0 | 20.0 |
| STEARETH-2 | 2.3 | 3.0 |
| STEARETH-21 | 1.5 | 1.0 |
| PERFUME | 1.0 | 1.0 |
| PPG-15 STEARYL ETHER | 0.5 | 2.0 |
| ALUMINUM STARCH OCTENYLSUCCINATE | — | — |
| ALLANTOIN | 0.1 | 0.1 |
| ISOPROPYL MYRISTATE | 0.1 | 0.1 |

The testers each applied an identical defined quantity of the roll-on emulsions of Examples 4, C1, or C2 onto the skin, and determined the time (in seconds) until the skin was once again perceived as dry.

Arithmetic means were calculated from the measured values for drying time, and are presented in the summary below:

| | Example 4 | C1 | C2 |
|---|---|---|---|
| Average drying time (seconds) | 340 | 470 | 520 |

In a further test using five test subjects, a variety of polysaccharides were compared with one another. Two formulations having a high ethanol content were also tested.

For this, 0.15 g of product was applied with a 1-ml syringe onto the underarm, and then distributed with a finger over a distance of 10 cm. The testers then determined the time (in seconds) until the skin was once again perceived as dry. The values are summarized in the table below.

TABLE
Drying time measurements.

| Formula | Tester 1 | Tester 2 | Tester 3 | Tester 4 | Tester 5 | Avg. drying time | |
|---|---|---|---|---|---|---|---|
| code | (sec) | (sec) | (sec) | (sec) | (sec) | (sec) | Notes |
| 43/01 | 605 | 230 | 220 | 600 | 240 | 379 | Only very slight whitening |
| | | | | | | | on application |
| 43/02 | 625 | 245 | 245 | 350 | 180 | 329 | Whitens on application, |
| | | | | | | | powdery |
| 43/03 | 675 | 200 | 320 | 540 | 240 | 395 | |
| 43/04 | (945) | 180 | 300 | 615 | 150 | (438) 326 (not | Whitens on application |
| | | | | | | incl. tester 1) | |
| 43/05 | 390 | 350 | 310 | 450 | 230 | 346 | Whitens slightly on |
| | | | | | | | application |
| 43/06 | 350 | 205 | 350 | 505 | 240 | 330 | Very pleasant skin feel |
| 97/01 | 645 | 150 | 580 | 610 | 365 | 470 | Heavy white residue |
| 11/01 | 860 | 165 | 530 | 705 | 380 | 528 | Whitens on application |
| 32/07 | 125 | 205 | 210 | 120 | 300 | 193 | |
| 16/02 | 140 | 220 | 100 | 140 | 170 | 148 | |

The use of 0.1 to 0.3 wt % polysaccharide very considerably reduced the “felt” drying time as compared with the polysaccharide-free formulations 97/01 and 11/01.

Formulations 32/07 and 16/02 are quick-drying, which was attributable by one skilled in the art to the high ethanol content. Proceeding therefrom, an influence on the “felt” drying time due to the polysaccharide content was not to be expected by one skilled in the art.

Formulations Used (Quantities Indicated in wt %).

| | Code | Code | Code | Code | Code | Code | Code | Code | Code | Code |
|---|---|---|---|---|---|---|---|---|---|---|
| | 43/01 | 43/02 | 43/03 | 43/04 | 43/05 | 43/06 | 97/01 | 11/01 | 32/07 | 16/02 |
| AQUA | 75.45 | 75.25 | 75.45 | 75.25 | 74.35 | 74.35 | 74.30 | 72.70 | 44.5441 | 44.5445 |
| STEARETH-2 | 2.40 | 2.40 | 2.40 | 2.40 | 2.40 | 2.40 | 2.40 | 3.00 | — | — |
| PPG-15 | 0.50 | 0.50 | 0.50 | 0.50 | 0.50 | 0.50 | 0.50 | 2.00 | — | — |
| STEARYL ETHER | | | | | | | | | | |
| STEARETH-21 | 1.50 | 1.50 | 1.50 | 1.50 | 1.50 | 1.50 | 1.50 | 1.00 | — | — |
| ALUMINUM | 20.00 | 20.00 | 20.00 | 20.00 | 20.00 | 20.00 | 20.00 | 20.00 | 20.00 | 17.00 |
| CHLORO- | | | | | | | | | | |
| HYDRATE | | | | | | | | | | |
| ALLANTOIN | — | — | — | — | — | — | 0.10 | 0.10 | — | — |
| ISOPROPYL | — | — | — | — | — | — | 0.10 | 0.10 | — | — |
| MYRISTATE | | | | | | | | | | |
| PERFUME | — | — | — | — | 1.10 | 1.10 | 1.10 | 1.10 | — | — |
| NaOH | — | — | — | — | — | — | — | — | 0.0009 | 0.0005 |
| ETHANOL 96% | — | — | — | — | — | — | — | — | 30.00 | 33.00 |
| CETEARETH-12 | — | — | — | — | — | — | — | — | 2.00 | 2.00 |
| CETEARETH-30 | — | — | — | — | — | — | — | — | 2.00 | 2.00 |
| BIS-PEG-18 | — | — | — | — | — | — | — | — | 1.00 | 1.00 |
| METHYL ETHER | | | | | | | | | | |
| DIMETHYL | | | | | | | | | | |
| SILANE | | | | | | | | | | |
| TOCOPHE-RYL | 0.05 | 0.05 | 0.05 | 0.05 | 0.05 | 0.05 | — | — | 0.05 | 0.05 |
| ACETATE | | | | | | | | | | |
| ALOE POWDER | — | — | — | — | — | — | — | — | 0.005 | 0.005 |
| HYDROXY- | — | — | — | — | — | — | — | — | 0.40 | 0.40 |
| ETHYL | | | | | | | | | | |
| CELLULOSE | | | | | | | | | | |
| DISTARCH | 0.10 | 0.30 | — | — | 0.10 | — | — | — | — | — |
| PHOSPHATE | | | | | | | | | | |
| ALUMINUM | — | — | 0.10 | 0.30 | — | 0.10 | — | — | — | — |
| STARCH | | | | | | | | | | |
| OCTENYL- | | | | | | | | | | |
| SUCCINATE | | | | | | | | | | |

1. A cosmetic oil-in-water emulsion that does not represent a microemulsion, containing
a) 0.5 to 6.5 wt % oil phase or fat phase, encompassing at least one oil component that is liquid at 20° C., selected from
i) linear and branched saturated mono- or polyvalent C₃ to C₃₀ alkanols that are etherified with at least one propylene oxide unit per molecule,
ii) propylene glycol monoesters of branched saturated C₆ to C₃₀ alkanecarboxylic acids,
iii) branched saturated C₁₀ to C₃₀ alkanols,
b) at least 60 wt % water,
c) 0.00001 to 38 wt % of at least one cosmetic active substance selected from
perspiration-inhibiting active substances,
deodorizing active substances,
monomers, oligomers, and polymers of amino acids, N—C₂-C₂₄ acylamino acids, the esters and/or the physiologically compatible salts of said substances,
DNA or RNA oligonucleotides,
natural betaine compounds,
vitamins, provitamins, and vitamin precursors of groups A, B, C, E, H, and K, and the esters of the aforesaid substances,
α-hydroxycarboxylic acids, α-ketocarboxylic acids, β-hydroxycarboxylic acids, and their ester, lactone, or salt form,
flavonoids and flavonoid-rich plant extracts,
isoflavonoids and isoflavonoid-rich plant extracts,
polyphenols and polyphenol-rich plant extracts,
ubiquinone and ubiquinol, and derivatives thereof,
silymarin,
ectoin,
repellents,
inorganic and organic UV-filtering substances,
self-tanning active substances,
skin-lightening active substances,
skin-calming active substances,
moisture-donating active substances,
sebum-regulating active substances,
d) at least one polysaccharide,
all the quantitative indications being based on the total weight of the emulsion.

2. The cosmetic oil-in-water emulsion according to claim 1, wherein the at least one polysaccharide is selected from anionic and nonionic polysaccharides and mixtures thereof.

3. The cosmetic oil-in-water emulsion according to claim 1, wherein the anionic polysaccharide is selected from aluminum starch octenylsuccinate, sodium starch octenylsuccinate, calcium starch octenylsuccinate, distarch phosphates, hydroxyethyl starch phosphates, hydroxypropyl starch phosphates, sodium carboxymethyl starches, sodium starch glycolate, and mixtures thereof.

4. The cosmetic oil-in-water emulsion according to claim 1, wherein the nonionic polysaccharide is selected from starches, starch hydrolysates, cellulose, methyl cellulose, hydroxypropyl cellulose, hydroxyethyl cellulose, hydroxypropylmethyl cellulose, hydroxypropylethyl cellulose, hydroxyethylmethyl cellulose, and mixtures thereof.

5. The cosmetic oil-in-water emulsion according to claim 1, wherein the at least one polysaccharide is contained in a total quantity from 0.01 to 1.0 wt % based on the total weight of the emulsion.

6. The cosmetic oil-in-water emulsion according to claim 1, wherein the oil component i) is selected from addition products of at least six propylene oxide units per molecule to mono- or polyvalent C₃₋₃₀ alkanols, in particular to butanol, butanediol, myristyl alcohol, and stearyl alcohol.

7. The cosmetic oil-in-water emulsion according to claim 1, wherein the oil component i) is selected from PPG-13 butyl ether, PPG-14 butyl ether, PPG-9 butyl ether, PPG-10 butanediol, and PPG-15 stearyl ether, and mixtures thereof.

8. The cosmetic oil-in-water emulsion according to claim 1, wherein the oil component ii) is selected from propylene glycol monoisostearate, propylene glycol monoisopalmitate, propylene glycol monoisobehenate, propylene glycol monoisoarachidate, propylene glycol monoisomyristate, propylene glycol monoisocaprate, propylene glycol monoisocaprinate, and propylene glycol monoisocaprylate, and mixtures thereof.

9. The cosmetic oil-in-water emulsion according to claim 1, wherein the oil component iii) is selected from isostearyl alcohol, isocetyl alcohol, isomyristyl alcohol, isotridecyl alcohol, isoarachidyl alcohol, isobehenyl alcohol, isocapryl alcohol, isocaprinyl alcohol, isocaprylyl alcohol, and mixtures thereof.

10. The cosmetic oil-in-water emulsion according to claim 1, which further contains at least one nonionic emulsifier having an HLB value in the range from 3 to 6.

11. The cosmetic oil-in-water emulsion according to claim 10, wherein the at least one nonionic emulsifier having an HLB value in the range from 3 to 6 is selected from linear saturated and unsaturated C₁₂ to C₃₀ alkanols that are etherified with 1 to 4 ethylene oxide units per molecule.

12. The cosmetic oil-in-water emulsion according to claim 11, wherein the at least one nonionic emulsifier having an HLB value in the range from 3 to 6 is selected from Steareth, Ceteth, Myristeth, Laureth, Trideceth, Arachideth, and Beheneth, having respectively 1 to 4 ethylene oxide units per molecule.

13. The cosmetic oil-in-water emulsion according to claim 10, wherein at least one nonionic emulsifier having an HLB value in the range from 3 to 6 is contained in a total quantity from 1.8 to 3 wt %, based on the weight of the entire emulsion.

14. The cosmetic oil-in-water emulsion according to claim 1, which further contains at least one nonionic emulsifier having an HLB value in the range from 12 to 18 is contained.

15. The cosmetic oil-in-water emulsion according to claim 14, wherein the at least one nonionic emulsifier having an HLB value in the range from 12 to 18 is selected from linear saturated and unsaturated C₁₂ to C₂₄ alkanols that are etherified with 7 to 40 ethylene oxide units per molecule.

16. The cosmetic oil-in-water emulsion according to claim 14, wherein the at least one nonionic emulsifier having an HLB value in the range from 12 to 18 is selected from Steareth, Ceteth, Myristeth, Laureth, Trideceth, Arachideth, and Beheneth, having respectively 7 to 40 ethylene oxide units per molecule.

17. The cosmetic oil-in-water emulsion according to claim 16, which further contains Steareth-2 as a nonionic emulsifier having an HLB value in the range from 3 to 6, and contains Steareth-21 as a nonionic emulsifier having an HLB value in the range from 12 to 18.

18. The cosmetic oil-in-water emulsion according to claim 17, which further contains PPG-15 stearyl ether.

19. A cosmetic oil-in-water emulsion that does not represent a microemulsion, containing
e) 0.5 to 6.5 wt % oil phase or fat phase, encompassing at least one oil component that is liquid at 20° C., selected from
iv) linear and branched saturated mono- or polyvalent C₃ to C₃₀ alkanols that are etherified with at least one propylene oxide unit per molecule,
v) propylene glycol monoesters of branched saturated C₆ to C₃₀ alkanecarboxylic acids,
vi) branched saturated C₁₀ to C₃₀ alkanols,
f) at least 60 wt % water,
g) 0.00001 to 38 wt % of at least one cosmetic active substance selected from
perspiration-inhibiting active substances,
deodorizing active substances,
monomers, oligomers, and polymers of amino acids, N—C₂-C₂₄ acylamino acids, the esters and/or the physiologically compatible salts of said substances,
DNA or RNA oligonucleotides,
natural betaine compounds,
vitamins, provitamins, and vitamin precursors of groups A, B, C, E, H, and K, and the esters of the aforesaid substances,
α-hydroxycarboxylic acids, α-ketocarboxylic acids, β-hydroxycarboxylic acids, and their ester, lactone, or salt form,
flavonoids and flavonoid-rich plant extracts,
isoflavonoids and isoflavonoid-rich plant extracts,
polyphenols and polyphenol-rich plant extracts,
ubiquinone and ubiquinol, and derivatives thereof,
silymarin,
ectoin,
repellents,
inorganic and organic UV-filtering substances,
self-tanning active substances,
skin-lightening active substances,
skin-calming active substances,
moisture-donating active substances,
sebum-regulating active substances,
h) at least one polysaccharide selected from aluminum starch octenyl succinate,
all the quantitative indications being based on the total weight of the emulsion.

20. The cosmetic oil-in-water emulsion according to claim 14, wherein at least one nonionic emulsifier having an HLB value in the range from 12 to 18 is present in a total quantity from 1 to 2 wt %, based on the weight of the entire emulsion.

21. The cosmetic oil-in-water emulsion according to claim 12, wherein the weight ratio of nonionic emulsifiers having an HLB value in the range from 3 to 6, and nonionic emulsifiers having an HLB value in the range from 12 to 18, is from 0.9 to 3.

22. The cosmetic oil-in-water emulsion according to claim 1, which further contains at most 3 wt %, based on the weight of the entire emulsion, of monovalent C₁ to C₃ alkanols.

23. The cosmetic oil-in-water emulsion according to claim 1, having a viscosity of from 1,000 to 5,000 mPas, measured one day after manufacture at 20° C. ambient temperature and 20° C. sample temperature.

24. A container having a ball applicator or roll-on applicator which contains a cosmetic oil-in-water emulsion that does not represent a microemulsion, containing
a) 0.5 to 6.5 wt % oil phase or fat phase, encompassing at least one oil component that is liquid at 20° C., selected from
vii) linear and branched saturated mono- or polyvalent C₃ to C₃₀ alkanols that are etherified with at least one propylene oxide unit per molecule,
viii) propylene glycol monoesters of branched saturated C₆ to C₃₀ alkanecarboxylic acids,
ix) branched saturated C₁₀ to C₃₀ alkanols,
i) at least 60 wt % water,
j) 0.00001 to 38 wt % of at least one cosmetic active substance selected from
perspiration-inhibiting active substances,
deodorizing active substances,
monomers, oligomers, and polymers of amino acids, N—C₂-C₂₄ acylamino acids, the esters and/or the physiologically compatible salts of said substances,
DNA or RNA oligonucleotides,
natural betaine compounds,
vitamins, provitamins, and vitamin precursors of groups A, B, C, E, H, and K, and the esters of the aforesaid substances,
α-hydroxycarboxylic acids, α-ketocarboxylic acids, β-hydroxycarboxylic acids, and their ester, lactone, or salt form,
flavonoids and flavonoid-rich plant extracts,
isoflavonoids and isoflavonoid-rich plant extracts,
polyphenols and polyphenol-rich plant extracts,
ubiquinone and ubiquinol, and derivatives thereof,
silymarin,
ectoin,
repellents,
inorganic and organic UV-filtering substances,
self-tanning active substances,
skin-lightening active substances,
skin-calming active substances,
moisture-donating active substances,
sebum-regulating active substances,
k) at least one polysaccharide,
all the quantitative indications being based on the total weight of the emulsion.

25. A process for perspiration-inhibiting treatment of skin, in particular, the armpit skin and/or foot skin, comprising the step of applying to the skin a cosmetic oil-in-water emulsion according to claim 1, in which the cosmetic active substance c) is selected from perspiration-inhibiting active substances.

26. A cosmetic, non-therapeutic method for perspiration-inhibiting treatment of the skin, in particular the armpit skin and/or foot skin, wherein an oil-in-water emulsion according to claim 1, in which the cosmetic active substance c) is selected from perspiration-inhibiting active substances, is applied in an effective quantity onto the skin.
